SEQUENCE LISTING (9 sequences)

SEQ ID NO: 1 - DNA, 13 bp, Artificial Sequence
  Binding oligonucleotide
tggggattcccca

SEQ ID NO: 2 - protein, 86 aa, Artificial Sequence
  Artificial Peptide
SPTSILDIKQGPKEPFRDYVDRFYKTLRAEQASQEVKNWMTETLLVQNANPDCKTILKALGPAATLEEMMTACQGVGGPGHKARVL

SEQ ID NO: 3 - protein, 86 aa, Artificial Sequence
  Artificial Peptide
NPTNILDIKQGPKEPFQSYVDRFYKSLRAEQTDPAVKNWMTQTLLIQNANPDCKLVLKGLGMNPTLEEMLTACQGVGGPGQKARLM

SEQ ID NO: 4 - protein, 86 aa, Artificial Sequence
  Artificial Peptide
NPVNILDIKQGPKEPFQSYVDRFYKSLRAEQADPAVKNWMTQTPLIQNANPDCKLVLKGLGMNPTLEEMLTACQGVGGPGQKARLM

SEQ ID NO: 5 - protein, 85 aa, Artificial Sequence
  Artificial Peptide
DPSWASILQGLEEPYHAFVERLNIALDNGLPEGTPKDPILRSLAYSNANKECQKLLQARGHTNSPLGDMLRACQTWTPKDKTKVL

SEQ ID NO: 6 - protein, 87 aa, Artificial Sequence
  Artificial Peptide
DPGASLTGVKQGPDEPFADFVHRLITTAGRIFGSAEAGVDYVKQLAYENANPACQAAIRPYRKKTDLTGYILCSDIGPSYQQGLAMA

SEQ ID NO: 7 - protein, 82 aa, Artificial Sequence
  Artificial Peptide
LAGLKQGNEESYETFISRLEEAVYRMMPRGEGSDILIKQLAWENANSLCQDLIRPIRKTGTIQDYIRACLDASPAVVQGMAY

SEQ ID NO: 8 - protein, 87 aa, Artificial Sequence
  Artificial Peptide
TNLAKVKGITQGPNESPSAFLERLKEAYRRYTPYDPEDPGQETNVSMSFIWQSAPDIGRKLERLEDLRNKTLGDLVREAERIFNKRE

SEQ ID NO: 9 - protein, 93 aa, Artificial Sequence
  Artificial Peptide
EPTDPWADIMQGPSESFVDFANRLIKAVEGSDLPPSARAPVIIDCFRQKSQPDIQQLIRAAPSTLTTPGEIIKYVLDRQKTAPLTDQGIAAAM

(19) United States
(12) Patent Application Publication
(10) Pub. No.: US 2003/0050242 A1
(43) Pub. Date: Mar. 13, 2003

(54) Protein polymerization inhibitors and methods of use

(76) Inventor: Anders Vahlne, Hovas (SE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/072,783

(22) Filed: Feb. 8, 2002

Related U.S. Application Data
(63) Continuation of application No. PCT/IB00/00972, filed on Jun. 29, 2000.
(60) Provisional application No. 60/147,981, filed on Aug. 9, 1999.

(51) Int. Cl. A61K 38/17; A61K 38/06

(52) U.S. Cl. 514/12; 514/18

(57) ABSTRACT

The present invention is related to the discovery of peptides that modulate the protein-protein interactions necessary for protein polymerization and the assembly of supramolecular protein complexes. More specifically, biotechnological tools and medicaments comprising various small peptides that have a modified carboxyl terminus are disclosed for use in the study and treatment or prevention of human disease.

### CROSS-REFERENCE TO RELATED APPLICATIONS

This application is a continuation of international application number PCT/IB00/00972, and claims the benefit of priority of international application number PCT/IB00/00972 having international filing date of Jun. 29, 2000, designating the United States of America and published in English, which claims the benefit of priority of U.S. provisional patent application No. 60/147,981, filed Aug. 9, 1999; both of which are hereby expressly incorporated by reference in their entireties.

### FIELD OF THE INVENTION

The present invention is related to the discovery of peptides that modulate the protein-protein interactions necessary for protein polymerization and the assembly of supramolecular protein complexes. More specifically, biotechnological tools and medicaments comprising various small peptides that have a modified carboxyl terminus are disclosed for use in the study and treatment or prevention of human disease.

### BACKGROUND OF THE INVENTION

Supramolecular structures such as transcription complexes, bacterial toxins, protein filaments and bundles, and viral protein coats are formed by the non-covalent assembly of many molecules, called “subunits”. Protein-protein interactions between the subunits stabilize these complexes and provide structural integrity. This process is evolutionarily favored because the building of a large structure from smaller subunits provides a highly diverse population of complexes from the least amount of genetic information, the assembly and disassembly of such structures can be readily controlled (since the subunits associate through multiple bonds of relatively low energy), and errors in the synthesis of the structure can be more easily avoided since correction mechanisms can operate during the course of assembly to exclude malformed subunits. (See, Alberts et al., Molecular Biology of the Cell, Third Edition, Garland Publishing, Inc., New York and London, pp. 123 (1994)).

Many proteins and protein complexes that regulate gene expression (e.g. transcriptional activators and repressors) achieve a strong interaction with a nucleic acid through protein-protein interactions and protein polymerization. In a simple case, one subunit associates with another subunit to form a dimer. Protein-protein interactions between the two monomers stabilize the dimer. Helix-turn-helix proteins, for example, are a family of proteins that comprise hundreds of DNA-binding proteins that bind as symmetric dimers to DNA sequences that are composed of two very similar “half-sites,” which are also arranged symmetrically. This arrangement allows each protein monomer to make a nearly identical set of contacts and enormously increases binding affinity. A second important group of DNA-binding motifs utilizes one or more molecules of zinc as a structural component. Such zinc-coordinated DNA-binding motifs, call zinc fingers, also form dimers that allow one of the two α helices of each subunit to interact with the major groove of the DNA. Further, a third protein motif, called the leucine zipper motif, recognizes DNA as a dimer. In leucine zipper domains, two α helices, one from each monomer, are joined together to form a short coiled-coil. Gene regulatory proteins that contain a leucine zipper motif can form either homodimers, in which the two monomers are identical, or heterodimers in which the monomers are different. A fourth group of regulatory proteins that bind DNA as a dimer comprise a helix-loop-helix motif. As with leucine zipper proteins, helix-loop-helix proteins can form homodimers or heterodimers. (See, Alberts et al., Molecular Biology of the Cell, Third Edition, Garland Publishing, Inc., New York and London, pp. 124 (1994)). Many gene regulatory proteins, in particular transcription factors, depend on protein-protein interactions and protein polymerization to function properly.

Similarly, the function of several bacterial toxins depend on protein-protein interactions and the polymerization of subunits. For example, pertussis toxin, diptheria toxin, cholera toxin, Psuedomonas exotoxin A, the heat-labile toxin of E. coli, verotoxins, and shiga toxin have similar structures that are characterized by an enzymatically active A subunit that is polymerized to an oligomer of B subunits that are necessary for the formation of the holotoxin. (Stein et al., Nature, 355:748 (1992); Read et al., U.S. Pat. No. 5,856,122; Lingwood, Trends in Microbiology 4:147 (1996)). Many believe that the B subunits diverged from a common ancestral protein (e.g., a pentameric protein that recognizes cell-surface carbohydrates) and became associated with different enzymatic components. (Stein et al., Nature, 355:748 (1992)).

In addition to small supramolecular structures, large supramolecular complexes composed of multiple subunits are also present in nature. When mechanical strength is of major importance in a cell, molecular assemblies are usually made from fibrous rather than globular subunits. Whereas short coiled-coils serve as dimerization domains in several families of gene regulatory proteins, more commonly a coiled-coil will extend for more than 100 nm and serve as a building block for a large fibrous structure, such as the actin thick filaments or tubulin bundles. (Alberts et al., Molecular Biology of the Cell, Third Edition, Garland Publishing, Inc., New York and London, pp. 124-125 (1994)). The accumulation of large fibrous structures can be detrimental in some circumstances, however, and the unregulated deposition of polymerized proteins has been associated with various forms of cancer and amyloidosis-related neurodegenerative diseases, such as Alzheimer's disease and scrapie (prion-related disease).

Some protein subunits also assemble into flat sheets in which the subunits are arranged in hexagonal arrays. Specialized membrane proteins are frequently arranged in this way in lipid bilayers. With a slight change in geometry of individual subunits, a hexagonal sheet can be converted into a tube or, with more changes, into a hollow sphere. These principles are dramatically illustrated in the assembly of the protein capsid of many viruses. These coats are often made of hundreds of identical protein subunits that enclose and protect the viral nucleic acid. The protein in such a capsid has a particularly adaptable structure, since it makes several different kinds of contacts and also changes its arrangement to let the nucleic acid out to initiate viral replication once the virus has entered a cell. The information for forming many of the complex assemblies of macromolecules and cells is contained in the subunits themselves, since under appropriate conditions, isolated subunits spontaneously assemble into a final structure.

Many protein-protein interactions that are present in nature are essential for mediating protein function, protein polymerization, and supramolecular complex assembly. The association of transcription factors, transcription complexes, bacterial toxins, fibrous assemblies, and viral capsids depend on protein-protein interactions and protein polymerization. The discovery of agents that selectively inhibit these protein-protein interactions and protein polymerization events would enable the development of novel biotechnological tools, therapeutics, and prophylactics for the study, treatment, and prevention of numerous diseases.

### SUMMARY OF THE INVENTION

Embodiments of the present invention include modified small peptides (two to ten amino acids in length) that inhibit protein-protein interactions, protein polymerization, and the assembly of supramolecular complexes. The selection, design, manufacture, characterization, and use of such peptide agents termed protein polymerization inhibitors, are collectively referred to as “PPI Technology”. The use of PPI technology can extend to many areas including but not limited to biotechnological research and development, as well as, therapeutic and prophylactic medicine.

Many biochemical events (e.g., the formation of transcription factor dimers, transcription complexes, bacterial toxins, and fibrous or bundled structures, and viral capsid assembly) depend on protein-protein interactions that assemble protein subunits into protein polymers and complexes. A way to disrupt assembly of such supramolecular structures, that for their particular function are dependent on di-, tri-, tetra-, or polymerization, is to construct small molecules that affect such protein-protein interactions, protein polymerization, and complex assemblies. It was discovered that small peptides with their carboxyl terminus hydroxyl group replaced with an amide group have such an inhibiting effect. Thus, embodiments of the present invention include to modified small peptides that effect protein-protein interactions, protein polymerization, and the assembly of protein complexes.

In desirable embodiments, the modified short peptides bind to a protein at a region that is involved in a protein-protein interaction and/or subunit assembly and thereby inhibit or prevent protein polymerization or the formation of a protein complex. In some embodiments, small peptides, which have a sequence that corresponds to a sequence of a transcription factor, interact with monomers of the transcription factor and prevent dimerization. In other embodiments, small peptides that have a sequence that corresponds to a transcriptional activator or repressor interact with the protein and modulate the assembly of a transcription activator or repressor complex. The NF-κB/IκB complex, for example, is unable to activate transcription, however, small peptides that interact with NF-κB or IκB, at regions involved in the protein-protein interactions that stabilize the complex, can modulate complex formation (e.g., inhibit or prevent or enhance) so as to enhance gene expression or prevent or retard gene expression. Methods are provided to modulate the assembly of the NF-κB and IκB complex by administering small peptides having a sequence that corresponds to regions of protein-protein interaction that are involved in the assembly or stabilization of the complex. Further, methods to identify small peptides that modulate the assembly of the NF-κB and IκB complex are provided. The small peptides identified for their ability to modulate the assembly of the NF-κB and IκB complex can be used as biotechnological tools or can be administered to treat or prevent diseases associated with an aberrant regulation of the NF-κB and IκB complex.

In other embodiments, modified small peptides that correspond to sequence in a subunit of a bacterial toxin, such as pertussis toxin, diphtheria toxin, cholera toxin, Pseudomonas exotoxin A, the heat-labile toxin of E. coli, and verotoxin, are used to prevent or inhibit the assembly of a bacterial holotoxin. Methods are provided, for example, to inhibit or prevent the assembly and function of pertussis toxin by administering small peptides having a sequence that corresponds to regions of protein-protein interaction that are involved in the assembly or stabilization of the subunits that form the holotoxin. Further, methods to identify other small peptides that inhibit or prevent bacterial holotoxin assembly are provided. The small peptides identified for their ability to inhibit the formation of a bacterial holotoxin can be used as biotechnological tools or can be administered to treat or prevent the toxic effects of a bacterial holotoxin.

Additional embodiments include the manufacture and identification of small peptides that inhibit the polymerization of fibrous proteins, such as actin, β-amyloid peptides, and prion-related proteins. Methods are provided to inhibit or prevent the polymerization of actin, β-amyloid peptide, and prion-related proteins by administering modified small peptides having a sequence that corresponds to regions of protein-protein interaction that are involved in protein polymerization. Further, methods to identify small peptides that inhibit or prevent protein polymerization are provided. The small peptides identified for their ability to inhibit actin, β-amyloid peptide, and prion-related protein polymerization can be used as biotechnological tools or can be administered to treat or prevent diseases associated with an aberrant actin, β-amyloid peptide, or prion-related protein polymerization including neurodegenerative diseases such as Alzheimer's disease and scrapie.

Other aspects of the invention include the manufacture and identification of small peptides that inhibit the polymerization of tubulin. Inhibitors of tubulin polymerization have been administered for the treatment of various forms of cancer for several years but there remains a need for less toxic tubulin polymerization inhibitors. Small peptides that correspond to sequences of tubulin that are involved in tubulin polymerization can be administered orally with little or no side-effects. Methods are provided to inhibit or prevent tubulin polymerization by administering small peptides having a sequence that corresponds to regions of protein-protein interaction that are involved in tubulin polymerization. Further, methods to identify small peptides that modulate (e.g., inhibit, prevent or enhance) tubulin polymerization are provided. The small peptides identified for their ability to effect tubulin polymerization can be used as biotechnological tools or can be administered to treat or prevent diseases associated with an aberrant tubulin polymerization.

In preferred embodiments, modified small peptides that correspond to sequences involved in viral capsid assembly are used to disrupt protein-protein interactions and, thereby, inhibit or prevent viral capsid assembly. For example, the small peptides Gly-Pro-Gly-NH2 (GPG-NH₂), Gly-Lys-Gly-NH₂ (GKG-NH₂), Cys-Gln-Gly-NH₂ (CQG-NH₂), Arg-Gln-Gly-NH₂ (RQG-NH₂), Lys-Gln-Gly-NH₂ (KQG-NH₂), Ala-Leu-Gly-NH2 (ALG-NH₂), Gly-Val-Gly-NH₂ (GVG-NH₂), Val-Gly-Gly-NH₂ (VGG-NH₂), Ala-Ser-Gly-NH₂ (ASG-NH₂), Ser-Leu-Gly-NH₂ (SLG-NH₂), and Ser-Pro-Thr-NH₂ (SPT-NH₂) are the preferred species. Methods are provided to inhibit or prevent viral capsid assembly by administering small peptides having a sequence that corresponds to regions of protein-protein interaction that are involved in the assembly or stabilization of the viral capsid. Further, methods to identify small peptides that inhibit or prevent the assembly of viral capsid are provided. The small peptides identified for their ability to inhibit or prevent the assembly of a viral capsid can be used as biotechnological tools or can be administered to treat or prevent viral infections, such as HIV infection. Pharmaceuticals comprising the modified small peptides of the invention are disclosed and methods of preparing such pharmaceuticals, prophylactics, and therapeutics for the treatment and prevention of diseases associated with protein-protein interactions, protein polymerization, and the assembly of supramolecular complexes are provided.

### BRIEF DESCRIPTION OF THE DRAWINGS

FIG. 1 is a composite of electron micrographs of untreated HIV particles.

FIG. 2 is a composite of electron micrographs of HIV particles that have been contacted with the protease inhibitor Ritonavir.

FIG. 3 is a composite of electron micrographs of HIV particles that have been contacted with GPG-NH₂

FIG. 4 is a graph representing the results from an HIV infectivity study conducted in HUT78 cells.

FIG. 5 illustrates an alignment of the protein sequence corresponding to the carboxyl terminus of the HIV-1 p24 protein (residues 146-231) and protein sequences of HIV-2, SIV, Rous Sarcoma viraus (RSV), human T cell leukemia virus-type 1 (HTLV-1), mouse mammary tumor virus (MMTV), Mason-Pfizer monkey virus (MPMV), and Moloney murine leukemia virus (MMLV). The bar represents the major homology region(MHR).

### DETAILED DESCRIPTION OF THE PREFERRED EMBODIMENT

It has been discovered that modified small peptides having sequences that correspond to regions of protein-protein interaction prevent and/or inhibit protein polymerization and the assembly of supramolecular complexes. In many supramolecular structures, protein subunits (e.g., protein monomers) undergo an assembly or polymerization process, which involves non-covalent protein-protein interactions, to generate a polymer of protein molecules. Small peptides having an amide instead of a hydroxyl group at the carboxyl terminus interrupt this polymerization process by inhibiting the protein-protein interactions that are necessary for the generation of the polymer. Such small peptides, referred to as protein polymerization inhibitors are useful in the manufacture of biotechnological tools and pharmaceuticals for the study and prevention and treatment of human disease. Further, approaches to make biotechnological tools and pharmaceutical compositions comprising modified small peptides and/or peptidomimetics that resemble these small peptides (collectively referred to as “peptide agents”) that correspond to sequences of transcription factors, bacterial toxins, fibrous or bundled proteins, viral capsid proteins, and other proteins involved in protein polymerization and supramolecular assembly are given below.

In some embodiments, small peptides, which have a sequence that corresponds to a sequence of a transcriptional activator, interact with monomers of the transcription factor and prevent dimerization. By inhibiting dimerization of a transcriptional activator (e.g., NF-κB), the expression of genes activated by the transcription factor can be effectively reduced or inhibited. NF-κB consists of two proteins having molecular weights of 50 and 65 kD. NF-κB is thought to be a transcriptional regulator of gene expression for various cytokine genes. (Haskill et al., U.S. Pat. No. 5,846,714). Small peptides that correspond to sequence of NF-κB involved in the protein-protein interactions that stabilize the activator disrupt the complex and, thereby, inhibit the expression of cytokine genes. Such inhibitors have use as biotechnological tools and as pharmaceuticals (e.g., for the treatment of inflammatory diseases characterized by an overexpression of cytokine genes).

In other embodiments, small peptides that have a sequence that corresponds to a transcriptional activator or repressor interact with the transcription factor, modulate the assembly of a transcription repressor complex, and, thereby, regulate gene expression. As described above, NFκB is a transcriptional activator that binds to DNA regulatory regions of certain cytokine genes. (Haskill et al., U.S. Pat. No. 5,846,714). NF-κB is regulated by its association with a 36 kD repressor protein termed IκB. The complex of NF-κB and IκB (“NFκB/IκB”) is unable to activate transcription, however, when NFκB is phosphorylated, IκB dissociates and transcriptional activation can take place. Small peptides that interact with NF-κB or IκB, preferably at regions involved in the protein-protein interactions that stabilize the NF-κB/IκB complex, inhibit or prevent complex formation so as to enhance gene expression, or, alternatively, can stabilize the complex and, thus, prevent or retard gene expression. Many small peptides that modulate the association of NFκB to IκB can be identified by using the methods described below. As above, the small peptides identified for their ability to modulate the assembly of the NF-κB/IκB complex can be used as biotechnological tools or can be administered to treat or prevent diseases associated with an aberrant regulation of the NF-κB/IκB complex.

In other embodiments, methods of manufacture, identification, and use of small peptides for the inhibition of protein polymerization necessary for the assembly of bacterial toxins are provided. To be effective, bacterial toxins must deliver the catalytic subunit of the holotoxin to an appropriate interaction site. Several bacterial toxins have adpated to this problem by forming a supramolecular structure that comprises two functional components, a catalytic component and a cellular recognition or binding component. In pertussis toxin and verotoxin, for example, a catalytic subunit “A” is joined to a pentamer assembly comprised of five “B” subunits that are involved toxin binding. Modified small peptides that correspond to sequence in a subunit of a bacterial toxin, such as pertussis toxin, diphtheria toxin, Pseudomonas exotoxin A, the heat-labile toxin of E. coli, and verotoxin, can be used to prevent or inhibit the assembly of a bacterial holotoxin and, thereby, reduce or inhibit the toxicity of the bacterial toxin. Methods to identify other small peptides that inhibit bacterial holotoxin assembly are also provided below. The small peptides identified for their ability to inhibit the formation of a bacterial holotoxin can be used as biotechnological tools or can be administered to treat or prevent the toxic effects of a bacterial holotoxin.

Additionally, methods of manufacture and identification of small peptides that inhibit the polymerization of actin and β-amyloid peptides are within the scope of aspects of the present invention. β-amyloid deposition and aggregation or polymerization at a cell membrane has been shown to cause an influx of calcium, which causes nerve cell injury. This neuronal insult has been associated with several neurodegenerative diseases including, but not limited to, Alzheimer's, stroke, and Huntington's disease. Compounds that cause actin depolymerization, such as cytochalsins, are useful for maintaining calcium homeostasis despite the presence of polymerized β-amyloid peptides. Methods to identify small peptides that inhibit or prevent actin polymerization and β-amyloid peptide aggregation are described below. Small peptides that inhibit or prevent the polymerization of actin can be administered in conjunction with small peptides that inhibit or prevent the aggregation of β-amyloid peptides so as to restore calcium homeostasis and provide a therapeutically beneficial treatment for individuals afflicted with certain neurodegenerative diseases.

Other embodiments of the invention include the manufacture and identification of small peptides that inhibit the polymerization of tubulin. Inhibitors of tubulin polymerization, such as vinblastine and vincristine, have been administered for the treatment of various forms of cancer for several years but current tubulin polymerization inhibitors are associated with many side-effects and are not well received by the body. In contrast, small peptides that correspond to sequences of tubulin that are involved in polymerization can be administered orally with little or no side-effects and are well tolerated by the body. Methods to identify small peptides that inhibit the polymerization of tubulin are provided in the following disclosure. The small peptides, identified for their ability to inhibit the polymerization of tubulin, can be used as biotechnological tools or can be administered to treat or prevent cancer.

In some embodiments, methods of manufacture, identification, and use of modified small peptides that correspond to sequences on viral capsid proteins for the treatment and prevention of viral disease are provided. These small peptides bind to the viral capsid protein, inhibit viral capsid protein polymerization, and, thereby, inhibit viral infectivity. In vitro binding assays are used, for example, to demonstrate that small peptides having a sequence that corresponds to the viral capsid protein p24, bind to the major capsid protein (p24) of HIV-1. Further, by using electron microscopy, it is shown that the small peptides efficiently interrupt capsid protein polymerization and capsid assembly. Evidence that small peptides, such as GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂, inhibit the replication of HIV-1, HIV-2, and SIVis also provided.

Because the sequences of regions of several proteins involved in the protein-protein interactions that mediate protein polymerization and supramolecular assembly are known, several modified small peptides that correspond to these sequences can be selected, designed, manufactured, and rapidly screened to identify those that effectively inhibit and/or prevent protein binding or protein polymerization using the techniques described herein, or modifications of these assays as would be apparent to those of skill in the art given the present disclosure. Although preferable peptide agents are tripeptides having an amide group at their carboxy termini, such as GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂, compositions and methods of inhibiting protein-protein interactions and protein polymerization are provided, comprising a peptide in amide form having the formula X₁, X₂, X₃-NH₂ or the formula X₄, X₅, X₁, X₂, X₃-NH₂, wherein X₁, X₂, X₃, X₄, and X₅ are any amino acid and wherein any one or two amino acids can be absent. Desirable embodiments have a glycine residue as X₃.

In some embodiments, the peptide agents are provided in monomeric form; in others, the peptide agents are provided in multimeric form or in multimerized form. Support-bound peptide agents are also used in several embodiments. Pharmaceutical compositions comprising peptide agents are administered as therapeutics or prophylactics or both for the treatment and/or prevention of disease. In some embodiments, the pharmaceutical compositions comprising peptide agents are administered in combination with other conventional treatments for the particular disease.

The peptide agent is first selected and designed by a rational approach. That is, the peptide agent is selected and designed based on an understanding that the sequence of the peptide agent is involved in a protein-protein interaction that modulates protein polymerization or the assembly of a protein complex. Several pieces of information can aid in this selection process including, but not limited to, mutational analysis, protein homology analysis (e.g., analysis of other sequences that have related domains), protein modeling, and other approaches in rational drug design. Peptide agents can, of course, also be selected randomly.

The peptide agents are then manufactured using conventional peptide or chemical synthetic methods. Many peptide agents are also commercially available. Next, assays are performed that evaluate the ability of the peptide agent to bind to the protein of interest, interfere with the protein-protein interactions that enable protein polymerization and/or assembly of a supramolecular complex, and prevent disease. The assays described herein, which evaluate a peptide agent's ability to bind to a protein of interest, modulate protein polymerization or protein complex assembly, and prevent disease, are collectively referred to as “peptide agent characterization assays”. It should be understood that any number, order, or modification of the peptide agent characterization assays described herein can be employed to identify a peptide agent that modulates a protein-protein interaction, protein polymerization, or the assembly of a protein complex.

In the following, there are provided several software and hardware embodiments of the invention, as well as, computational methods that can be used to aid in the selection and design of the peptide agents of the invention.

Software and Hardware Embodiments

The nucleic acid sequence and/or the protein sequence of a polypeptide of interest or fragments thereof (e.g., a protein involved in a protein-protein interaction, protein polymerization, or the assembly of a protein complex) can be entered onto a computer readable medium for recording and manipulation. It will be appreciated by those skilled in the art that a computer readable medium having the nucleic acid sequence and the protein sequence of a protein of interest or fragments thereof is useful for the determination of homologous sequences, structural and functional domains, and the construction of protein models. The utility of a computer readable medium having the nucleic acid sequence and/or protein sequence of the protein of interest or fragments thereof includes the ability to compare the sequence, using computer programs known in the art, so as to perform homology searches, ascertain structural and functional domains and develop protein models so as to select peptide agents that modulate protein-protein interactions, protein polymerization, and the assembly of protein complexes.

The nucleic acid sequence and/or the protein sequence or fragments thereof of a protein involved in a protein-protein interaction, protein polymerization, or the assembly of a protein complex can be stored, recorded, and manipulated on any medium that can be read and accessed by a computer. As used herein, the words “recorded” and “stored” refer to a process for storing information on computer readable medium. A skilled artisan can readily adopt any of the presently known methods for recording information on computer readable medium to generate manufactures comprising the nucleotide or polypeptide sequence information of this embodiment.

A variety of data storage structures are available to a skilled artisan for creating a computer readable medium having recorded thereon a nucleotide or polypeptide sequence. The choice of the data storage structure will generally be based on the component chosen to access the stored information. Computer readable media include magnetically readable media, optically readable media, or electronically readable media. For example, the computer readable media can be a hard disc, a floppy disc, a magnetic tape, zip disk, CD-ROM, DVD-ROM, RAM, or ROM as well as other types of other media known to those skilled in the art. The computer readable media on which the sequence information is stored can be in a personal computer, a network, a server or other computer systems known to those skilled in the art.

Embodiments of the invention include systems, particularly computer-based systems that use the sequence and protein model information described herein to design and select peptide agents for the modulation of a protein-protein interaction, a protein polymerization event, or the assembly of a protein complex. The term “computer-based system” refers to the hardware, software, and any database used to analyze a polypeptide or sequence thereof for such purpose. The computer-based system preferably includes the storage media described above, and a processor for accessing and manipulating the sequence data. The hardware of the computer-based systems of this embodiment comprise a central processing unit (CPU) and a data database. A skilled artisan can readily appreciate that any one of the currently available computer-based systems are suitable.

In one particular embodiment, the computer system includes a processor connected to a bus which is connected to a main memory (preferably implemented as RAM) and a variety of secondary storage devices, such as a hard drive and removable medium storage device. The removable medium storage device may represent, for example, a floppy disk drive, a compact disk drive, a magnetic tape drive, etc. A removable storage medium, such as a floppy disk, a compact disk, a magnetic tape, etc. containing control logic and/or data recorded therein (e.g., nucleic acid sequence and/or the protein sequence or fragments thereof of a protein involved in a protein-protein interaction, protein polymerization, or the assembly of a protein complex) can be inserted into the removable storage device. The computer system includes appropriate software for reading the control logic and/or the data from the removable medium storage device once inserted in the removable medium storage device.

The nucleic acid sequence and/or the protein sequence or fragments thereof of a protein of interest can be stored in a well known manner in the main memory, any of the secondary storage devices, and/or a removable storage medium. Software for accessing and processing the nucleic acid sequence and/or the protein sequence or fragments thereof (such as search tools, compare tools, and modeling tools etc.) reside in main memory during execution.

As used herein, “a database” refers to memory that can store nucleotide or polypeptide sequence information, protein model information, and information on other peptides, chemicals, peptidomimetics, and other agents that modulate a protein-protein interaction, protein polymerization, or the assembly of a protein complex. Additionally, a “database” refers to a memory access component that can access manufactures having recorded thereon nucleotide or polypeptide sequence information, protein model information, and information obtained from the various peptide characterization assays provided herein. In some embodiments, a database stores the information described above for numerous peptide agents, and products so that a comparison of the data can be made. That is, databases can store this information as a “profile” for each peptide agent tested and profiles from different peptide agents can be compared so as to identify functional and structural characteristics that are needed in a derivative peptide agent to produce a desired response. Then these derivative molecules can be made by conventional techniques in molecular biology and protein engineering and tested in further rounds of functional assays. Additionally, profiles on numerous peptide agents are useful when developing strategies that employ multiple peptide agents. The use of multiple peptide agents (e.g., in a pharmaceutical for the treatment or prevention of disease) can modulate the association of the protein of interest with another protein or assemblage of proteins more effectively than administration of a peptide agent that modulates protein-protein interactions, protein polymerization, or proten complex formation at one site.

The sequence data of a protein of interest or a peptide agent or both can be stored and manipulated in a variety of data processor programs in a variety of formats. For example, the sequence data can be stored as text in a word processing file, such as MicrosoftWORD or WORDPERFECT, an ASCII file, a html file, or a pdf file in a variety of database programs familiar to those of skill in the art, such as DB2, SYBASE, or ORACLE. A “search program” refers to one or more programs that are implemented on the computer-based system to compare a nucleotide or polypeptide sequence of a protein of interest with other nucleotide or polypeptide sequences and the molecular profiles created as described above. A search program also refers to one or more programs that compare one or more protein models to several protein models that exist in a database and one or more protein models to several peptide agents, which exist in a database. A search program is used, for example, to compare regions of the protein sequence of a protein of interest or fragments thereof that match sequences in a data base having the sequences of peptide agents so as to identify corresponding or homologous sequences.

A “retrieval program” refers to one or more programs that are implemented on the computer based system to identify a homologous nucleic acid sequence, a homologous protein sequence, a homologous protein model, or a homologous peptide agent sequence.

A retrieval program is also used to identify peptides, peptidomimetics and chemicals that interact with a protein sequence, or a protein model stored in a database. Further a retrieval program is used to identify a profile from the database that matches a desired protein-protein interaction in a protein complex of interest.

In the discussion below, there are described several methods of molecular modeling, combinatorial chemistry, and rational drug design for the design and selection of peptide agents that interact with a protein of interest believed to be involved in a protein-protein interaction, protein polymerization, or the assembly of a protein complex.

Methods of Rational Drug Design

In some embodiments, search programs are employed to compare regions of a protein of interest to other proteins so that peptide agents that modulate protein-protein interactions, protein polymerization, or the assembly of a protein complex can be more efficiently selected and designed. In other embodiments, search programs are employed to compare regions of a protein of interest with peptide agents and profiles of peptide agents so that interactions of the peptide agent with the protein of interest (e.g., modulation of protein-protein interactions, protein polymerization, and the assembly of a protein complex) can be predicted. This process is referred to as “rational drug design”. Rational drug design has been used to develop HIV protease inhibitors and agonists for five different somatostatin receptor subtypes. (Erickson et al., Science 249:527-533 (1990) and Berk et al., Science 282:737 (1998)).

In one case, for example, the region of protein-protein interaction necessary for protein polymerization or protein complex assembly of a protein of interest is not known but such a region is known for a related protein. Starting with the sequence or a protein model of the protein of interest or fragments thereof, related or homologous polypeptides that have known regions of protein-protein interaction necessary for protein polymerization or subunit assembly can be rapidly identified. By comparing the known regions of protein-protein interaction in the newly found homologous protein to the protein of interest, domains of the protein of interest that are likely involved in protein-protein interaction can be identified and peptide agents that correspond to these regions can be selected and designed.

Accordingly, by a two-dimensional approach, a percent sequence identity can be determined by standard methods that are commonly used to compare the similarity and position of the amino acid of two polypeptides. Using a computer program such as BLAST or FASTA, two polypeptides are aligned for optimal matching of their respective amino acids (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program. The percent identity can then be calculated as:

$$\frac{total number of identical matches}{\begin{matrix}[ length of the longer sequence within the matched span + \\ number of gaps introduced into the longer \\ sequence in order to align the two sequences ]\end{matrix}} \times 100$$

The protein sequence of the protein of interest is compared to known sequences on a protein basis. The protein sequence of the protein of interest are compared, for example, to known amino acid sequences found in Swissprot release 35, PIR release 53 and Genpept release 108 public databases using BLASTP with the parameter W=8 and allowing a maximum of 10 matches. In addition, the protein sequence encoding the protein of interest is compared to publicly known amino acid sequences of Swissprot using BLASTX with the parameter E=0.001. Once a group of related polypeptides are identified, the available literature on the related protein sequences is reviewed so as to identify one or more related proteins, in which the protein-protein interactions that allow for protein polymerization and protein complex assembly have been determined. As the regions of a related protein that are involved in a protein-protein interaction, protein polymerization, or the assembly of a protein complex are realized, these sequences are compared to the protein of interest for homology, keeping in mind conservative amino acid replacements. In this manner, previously unknown regions of a protein of interest that are involved in protein-protein interactions, protein polymerization, and protein complex assembly can be determined and this information can be used to select and design peptide agents.

In addition, when the regions of protein-protein interaction necessary for protein polymerization, and protein complex assembly is not known, various techniques in mutational analysis can be employed to determine the domains of the protein necessary for subunit association. One technique is alanine scan (Wells, Methods in Enzymol. 202:390-411 (1991)). By this approach, each amino acid residue in a protein of interest is replaced by alanine, one mutant at a time, and the effect of each mutation on the ability of the protein to entertain a protein-protein interaction, a protein polymerization event, or participate in the assembly of a protein complex is measured. Each of the amino acid residues of the protein of interest is analyzed in this manner and the regions of the that have residues that are necessary for subunit association or polymerization are identified.

It is also possible to isolate a target-specific antibody, selected by its ability to modulate a protein-protein interaction necessary for protein polymerization or protein complex assembly, and solve its crystal structure so as to identify a region of the protein of interest amenable to modulation by a peptide agent. In principal, this approach yields a pharmacore upon which subsequent design can be based. By this approach, protein crystallography of the protein of interest is by-passed altogether by generating anti-idiotypic antibodies (anti-ids) to a functional, pharmacologically active antibody. As a mirror image of a mirror image, the binding site of the anti-ids would be expected to be an analog of a region of the protein of interest. The anti-id can then be used to design and select peptide agents.

Additionally, a three-dimensional structure of a protein of interest can be used to identify regions of the protein that are involved in a protein-protein interactions, protein polymerization, or the assembly of a protein complex. In the past, the three-dimensional structures of proteins have been determined in a number of ways. Perhaps the best known way of determining protein structure involves the use of x-ray crystallography. A general review of this technique can be found in Van Holde, K. E. Physical Biochemistry, Prentice-Hall, N.J. pp. 221-239 (1971). Using this technique, it is possible to elucidate three-dimensional structure with good precision. Additionally, protein structure may be determined through the use of techniques of neutron diffraction, or by nuclear magnetic resonance (NMR). (See, e.g., Moore, W. J., Physical Chemistry, 4^{th} Edition, Prentice-Hall, N.J. (1972)).

Alternatively, protein models can be constructed using computer-based protein modeling techniques. By one approach, the protein folding problem is solved by finding target sequences that are most compatible with profiles representing the structural environments of the residues in known three-dimensional protein structures. (See, e.g., Eisenberg et al., U.S. Pat. No. 5,436,850 issued Jul. 25, 1995). In another technique, the known three-dimensional structures of proteins in a given family are superimposed to define the structurally conserved regions in that family. This protein modeling technique also uses the known three-dimensional structure of a homologous protein to approximate the structure of a polypeptide of interest. (See e.g., Srinivasan, et al., U.S. Pat. No. 5,557,535 issued Sep. 17, 1996). Conventional homology modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods and “fuzzy” approaches now enables the identification of likely folding patterns and functional protein domains in a number of situations where the structural relatedness between target and template(s) is not detectable at the sequence level. By one method, fold recognition is performed using Multiple Sequence Threading (MST) and structural equivalences are deduced from the threading output using the distance geometry program DRAGON which constructs a low resolution model. A full-atom representation is then constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalences obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., Proteins:Structure, Function, and Genetics, Supplement 1:38-42 (1997)).

In one approach, a three-dimensional structure of a protein or a protein complex of interest is determined by x-ray crystallography, NMR, or neutron diffraction and computer modeling, as described above. Useful models of the protein or protein complex can also be gained by computer modeling alone. The regions of the protein(s) involved in a protein-protein interactions, protein polymerization, and the assembly of the protein complex are identified and peptide agents that correspond to these regions are selected and designed. The candidate peptide agents are then manufactured and tested in the peptide agent characterization assays described herein. Libraries of related peptide agents can be synthesized and these molecules are then screened in the peptide agent characterization assays. Compounds that produce desirable responses are identified, recorded on a computer readable media, (e.g., a profile is made) and the process is repeated to select for optimal peptide agents. Each newly identified peptide agent and its performance in the peptide agent characterization assay is recorded on a computer readable media and a database or library of profiles on various petide agents are generated. These profiles are used by researchers to identify important property differences between active and inactive molecules so that peptide agent libraries (e.g., for use in strategies employing multiple peptide agents) are enriched for molecules that have favorable characteristics.

Further, a three-dimensional model of a protein or protein complex of interest can be stored in a first database, a library of peptide agents that correspond to the protein or protein complex and their profiles can be stored in a second database, and a search program can be used to compare the model of the first database with the peptide agents of the second database given the parameters defined by the profiles of the peptide agents. A retrieval program can then be employed to obtain a peptide agent or a plurality of peptide agents that predictively modulate a protein-protein interaction, protein polymerization, or the assembly of a protein complex. Subsequently, these peptide agents can be screened in the peptide agent characterization assays. This technique can be extremely useful for the rapid selection and design of peptide agents and can be used to fabricate treatment protocols for human disease.

Many computer programs and databases can be used with embodiments of the invention to select and design peptide agents. The following list is intended not to limit the invention but to provide guidance to programs and databases which are useful with the approaches discussed above. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403 (1990)), FASTA (Pearson and Lipman, Proc. Natl. Acad. Sci. USA, 85: 2444 (1988)), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), Modeller 4 (Sali and Blundell J. Mol. Biol. 234:217-241 (1997)), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, and the BioByteMasterFile database. Many other programs and data bases would be apparent to one of skill in the art given the teachings herein.

Once a peptide agent has been selected and designed it can be manufactured by many approaches known in the art. Further, many commercial enterprises specialize in the manufacture of made-to-order peptides, peptidomimetics, and chemicals. The following discussion provides a general approach for the manufacture of the modified small peptides.

Obtaining the Peptide Agents

The approaches used to obtain the modified small peptides described herein are disclosed in this section. Several tripeptides that were used for the experiments disclosed herein were chemically synthesized with an automated peptide synthesizer (Syro, Multisyntech, Tubingen, Germany). The synthesis was run using 9-fluorenylmethoxycarbonyl (fmoc) protected amino acids (Milligen, Bedford, Mass.) according to standard protocols. All peptides were lyophilized and then disolved at the appropriate concentration in phosphate-buffered saline (PBS). The peptides were analyzed by reverse phase high performance liquid chromatography (RP-HPLC) using a PepS-15 C18 column (Pharmacia, Uppsala, Sweden).

In many embodiments, peptides having a modulation group attached to the carboxy-terminus of the peptide (“modified peptides”) were used. In some cases, the modified peptides were created by substituting an amino group for the hydroxyl residue normally present at the terminal carboxyl group of a peptide. That is, instead of a terminal COOH, the peptides were synthesized to have CO-NH₂. For example, preferred small peptides include glycyl-lysyl-glycine amide (GKG-NH₂), cystyl-glutaminyl-glycine amide (CQG-NH₂), glycyl-prolyl-glycine amide (GPG-NH₂), arginyl-glutaminyl-glycine amide (RQG-NH₂), lysyl-glutaminyl-glycine amide (KQG-NH₂), alanyl-leucyl-glycine amide (ALG-NH₂), glycyl-valyl-glycine amide (GVG-NH₂), valyl-glycyl-glycine amide (VGG-NH₂), alanyl-seryl-glycine amide (ASG-NH₂), seryl-leucyl-glycine amide (SLG-NH₂), and seryl-prolyl-threonine amide (SPT-NH₂). In addition to those synthesized, many tripeptides were also purchased from Bachem AG, Switzerland, including but not limited to, GKG-NH₂, CQG-NH₂, and GPG-NH₂.

There are many ways to synthesize small peptides, and the description above is provided as one possible way to obtain the modified small peptide embodiments disclosed herein. Several approaches to make peptidomimetics that resemble the small peptides described herein are known in the art. A vast number of methods, for example, can be found in U.S. Pat. Nos. 5,288,707; 5,552,534; 5,811,515; 5,817,626; 5,817,879; 5,821,231; and 5, 874,529, herein incorporated by reference in their entirety.

After the peptide agent has been selected, designed, and manufactured it is tested in one or more peptide characterization assays to determine the ability of the peptide agent to modulate a protein-protein interaction and/or protein polymerization and/or protein complex assembly. The peptide characterization assays can, for example, evaluate a peptide agent's ability to bind to a protein of interest, modulate protein polymerization or protein complex assembly, and prevent disease. Use of the peptide characterization assays to identify peptide agents for incorporation into biotechnological tools and pharmaceuticals is described below in reference to particular examples and applications. These examples and applications are not intended to limit the scope of the invention to the particular embodiments discussed because the technology described herein can be employed to modulate several other protein-protein interactions, protein polymerization events, and protein complex assemblies.

In the following, a description of the use of PPI technology to inhibit the dimerization of a transcriptional activator, NFκB, is provided.

Inhibition of Dimerization of a Transcriptional Activator

Members of the rel/NFκB family of transcription factors play a vital role in the regulation of rapid cellular responses, such as those required to fight infection or react to cellular stress. Members of this family of proteins form homo- and heterodimers with differing affinities for dimerization. They share a structural motif known as the rel homology region (RHR), the C-terminal one third of which mediates protein dimerization. (Huang et al., Structure 5:1427-1436 (1997)). Crystal structures of the rel/NFκB family members p50 and p65 in their DNA-bound homodimeric form have been solved. These structures showed that the residues from the dimerization domains of both p50 and p65 participate in DNA binding and that the DNA-protein and protein dimerization surfaces form one continuous overlapping interface. (Huang et al., Structure 5:1427-1436 (1997)). Further, the crystal structures of the dimerization domains of murine p50 and p65 at 2.2 Å and 2.0 Å resolution have been solved and a comparison of these two structures reveals that conservative amino acid changes at three positions are responsible for the differences in their dimer interfaces. Amino acids at positions corresponding to 254, 267, and 307 of murine p50, function as primary determinants for the observed differences in dimerization affinity. (Huang et al., Structure 5:1427-1436 (1997)).

The findings above can be used to select and design peptide agents that modulate NFκB dimerization. The crystal structure of murine p50 was used to determine that amino acid residues 254, 267, and 307 of p50 are involved in dimerization of NFκB. Peptide agents that correspond to overlapping sequences encompassing these amino acid residues can be designed, manufactured and screened in the peptide agent characterization assays. Additionally, the murine model of p50 can be compared with the human model of p50 to discern the region of the protein that corresponds to amino acid residues 254, 267, and 307. Because of the high degree of homology of the mouse and human NFκB p50 proteins, it is likely that amino acids residues 254, 267, and 307 or amino acids near these sites are necessary for dimerization of human NFκB. Further, peptide agents can be selected and designed to other regions of p50 and p65 and preferable peptide agents correspond to sequences found in the C-terminal-end of the rel homology region (RHR), which mediates protein dimerization. (Huang et al., Structure 5:1427-1436 (1997)).

Once the peptide agents that correspond to regions of p50 and p65 are selected, designed, and manufactured they are screened in peptide agent characterization assays. Initially, binding assays are conducted. By one approach, p50, p65, or the p105 dimer is placed in a dialysis membrane with a 10,000 mw cut-off (e.g., a Slide-A-lyzer, Pierce). Alternatively the protein of interest is immobilized on a support (e.g., an affinity chromatography resin or well of a microtiter plate). Radioactively labeled peptide agents are added in a suitable buffer and the binding reaction is allowed to take place overnight at 4° C. The peptide agents can be radiolabeled with ¹²⁵I or ¹⁴C, according to standard techniques or can be labeled with other detectable signals. After the binding reaction has taken place, the peptide agent-containing buffer is removed, and either the protein-bound support is washed in a buffer without radioactive peptide agents or the dialysis membrane having the protein of interest is dialyzed for two hours at 4° C. in a buffer lacking radioactive peptide agents. Subsequently, the radioactivity bound to the protein on the support or the radioactivity present in the dialyzed protein is measured by scintillation. Peptide agents that bind to p50, p65, or p105 can be rapidly identified in this manner. Modifications of these binding assays can be employed, as would be apparent to those of skill in the art, in particular binding assays, such as described above are readily amenable to high throughput analysis, for example, by binding the protein of interest to a microtiter plate and screening for the binding of fluorescently labeled peptide agents.

After the binding of one or more peptide agents is determined, an assay that evaluates the ability of the peptide agent to modulate dimerization of NFκB is employed. One such assay is a gel-shift assay. (See e.g., Haskill et al., U.S. Pat. No. 5,846,714). NFκB dimers bind to a specific regulatory DNA enhancer having the sequence TGGGGATTCCCCA (SEQ. ID. NO. 1) and radioactively labeled (e.g., ³²P) oligonucleotides having this sequence can be used to resolve complexes of NFκB and the oligonucleotide in a low percentage, nondenaturing polyacrylamide gel.

Accordingly, a gel-shift assay that evaluates the ability of a peptide agent to inhibit the dimerization of NFκB is accomplished as follows. Oligonucleotides having the NFκB enhancer sequence are radioactively labeled by conventional approaches. These oligonucleotides are incubated in the presence of varying concentrations of the candidate peptide agents and a nuclear extract having NFκB at 23° C. for 15 minutes. Typical binding conditions can include 10 μg nuclear extract, 10,000 cpm oligonucleotide probe, 10 mM Tris, pH 7.7, 50 mM NaCl, 0.5 mM EDTA, 1 mM DTT, 2 μg poly dI-dC and 10% glycerol in a final volume of 20 μl. The NFκB containing nuclear extracts can be obtained from various cell types but are preferably obtained from mitogen and phorbal ester induced Jurkat T-cells. After binding, the complexes are resolved on a 5% non-denaturing polyacrylamide gel formed in Tris/glycine/EDTA buffer as described by Baldwin, DNA & Protein Eng. Tech. 2:73-76 (1990). Electrophoresis is conducted for 2 hours at 20 mA, then the gel is autoradiographed overnight at −70° C. Because the dimer complex of NFκB joined to the labeled oligonucleotide can be resolved from any monomer (p50 or p65) that remains asociated with the complex after electrophoresis, the ability of a peptide agent to inhibit dimerization of NFκB can be rapidly determined. Preferably, the concentration of the different peptide agents is titrated over the course of several experiments to find an amount that satisfactorily inhibits the formation of NFκB dimers.

Additionally, the ability of the candidate peptide agents to inhibit NFκB transcriptional activation in cells can be determined by treating cells that have been transfected with a NFκB reporter construct with varying concentrations of the peptide agents. A NFκB reporter construct can comprise, for example, three or more enhancer sequences (e.g., TGGGGATTCCCCA (SEQ. ID. NO. 1)) joined to a minimal promoter and a reporter molecule (e.g., luciferase, chloramphenicol acetyl transferase, or green fluorescent protein). Such a reporter construct can be made using techniques in molecular biology. Preferably, the reporter construct is transfected into a cell line that can produce copius amount of NFκB upon stimulation with a mitogen and a phorbal ester, such as Jurkat cells. Candidate peptide agents can be screened by transfecting the reporter construct in cells that have been cultured in the presence of varying concentrations of the peptide agents. By comparing the levels of reporter signal detected in untreated control cells to peptide agent-treated cells, the ability of a particular peptide agent to inhibit NFκB mediated transcriptional activation can be determined. Preferably, peptide agents that comprise the amino acids at positions corresponding to 254, 267, and 307 of murine p50 and other amino acids of the C terminal portion of the rel homology region are selected, designed, manufactured, and assayed using the techniques described above. In this manner, peptide agents that inhibit NFκB activation can be identified for incorporation into a pharmaceutical for the treatment and/or prevention of NFκB-related diseases.

In the following, a description of the use of PPI technology to inhibit the association of NFκB with the IκB repressor is provided.

Inhibition of a transcriptional repressor complex The inhibition of a transcriptional repressor complex can also be accomplished using the PPI technology. For example, peptide agents that correspond to sequences of NFκB and IκB that are involved in protein-protein interactions that stabilize the NFκB/IκB complex can be selected, designed, manufactured, and screened in peptide characterization assays to identify peptide agents that effectively modulate assembly of the NFκB/IκB complex.

Accordingly, peptide agents are selected and designed to correspond to sequences that have been shown to be involved in stabilizing the NFκB/IκB complex. The ankyrin-repeat-containing domain and the carboxyl-terminal acidic tail/PEST sequence are regions of IκB found to be involved in binding to the 105 kDa NFκB heterodimer. (Latimer et al., Mol. Cell Biol., 18:2640 (1998) and Malek et al., J. Biol. Chem., 273:25427 (1998)). Additionally, the nuclear localization sequence, the dimerization domain, and the amino-terminal DNA binding domain of NFκB interact with IκB so as to stabilize the NFκB/IκB complex. (Malek et al., J. Biol. Chem., 273:25427 (1998)). Peptide agents that correspond to these regions are selected, designed, and manufactured Next, the candidate peptide agents are screened in peptide characterization assays that evaluate their ability to bind to NFκB or IκB, inhibit the formation of the NFκB/IκB complex, and inhibit IκB-mediated transcriptional repression. To evaluate the ability of a peptide agent to bind to either NFκB or IκB, an in vitro binding assay is performed. As described earlier, there are several types of in vitro binding assays that are known in the art and desirable approaches involve the binding of radiolabeled peptide agents to NFκB or IκB proteins disposed on a support or in a dialysis membrane. By one approach, NFκB or IκB proteins are disposed in a dialysis membrane having a 10,000 mw cut-off (e.g., a Slide-A-lyzer, Pierce) or the protein of interest is immobilized on a support (e.g., an affinity chromatography resin or well of a microtiter plate). Then, radioactively labeled peptide agents are added in a suitable buffer and the binding reaction is allowed to take place overnight at 4° C. The peptide agents can be radiolabeled with 125I or ⁴C, according to standard techniques or can be labeled with other detectable signals. After the binding reaction has taken place, the peptide agent-containing buffer is removed, and either the protein-bound support is washed in a buffer without radioactive peptide agents or the dialysis membrane having the protein of interest is dialyzed for two hours at 4° C. in a buffer lacking radioactive peptide agents. Subsequently, the radioactivity bound to the protein on the support or the radioactivity present in the dialyzed protein is measured by scintillation. Peptide agents that bind to NFκB or IκB can be rapidly identified in this manner. Modifications of these binding assays can be employed, as would be apparent to those of skill in the art, in particular binding assays, such as described above are readily amenable to high throughput analysis, for example, by binding the protein of interest to a microtiter plate and screening for the binding of fluorescently labeled peptide agents.

After the binding of one or more peptide agents is determined, an assay that evaluates the ability of the peptide agent to inhibit the formation of the NFκB/IκB complex is employed. One such assay is a gel-shift assay. (See e.g., Haskill et al., U.S. Pat. No. 5,846,714). NFκB dimers bind to a specific regulatory DNA enhancer having the sequence TGGGGATTCCCCA (SEQ. ID. NO. 1) and radioactively labeled (e.g., ³²P) oligonucleotides having this sequence can be used to resolve complexes of NFκB and the oligonucleotide in a low percentage, nondenaturing polyacrylamide gel.

Accordingly, a gel-shift assay that evaluates the ability of a peptide agent to inhibit the assembly of NFκB/IκB complexes is accomplished as follows. Oligonucleotides having the NFκB enhancer sequence are radioactively labeled by conventional approaches. These oligonucleotides are incubated in the presence of varying concentrations of the candidate peptide agents and a nuclear extract having NFκB and IκB at 23° C. for 15 minutes. Typical binding conditions can include 10%g nuclear extract, 10,000 cpm oligonucleotide probe, 10 mM Tris, pH 7.7, 50 mM NaCl, 0.5 mM EDTA, 1 mM DTT, 2 μg poly dI-dC and 10% glycerol in a final volume of 20 μl. The NFκB and IκB containing nuclear extracts can be obtained from various cell types but are preferably obtained from mitogen and phorbal ester induced Jurkat T-cells. After binding, the complexes are resolved on a 5% non-denaturing polyacrylamide gel formed in Tris/glycine/EDTA buffer as described by Baldwin, DNA & Protein Eng. Tech. 2:73-76 (1990). Electrophoresis is conducted for 2 hours at 20 mA, then the gel is autoradiographed overnight at −70° C. Because the dimer complex of NFκB joined to the labeled oligonucleotide can be resolved on the gel after electrophoresis and NFκB/IκB complexes are unable to bind to the enhancer, the ability of a peptide agent to disrupt or prevent the formation of NFκB/IκB complexes can be rapidly determined. Preferably, the concentration of the different peptide agents is titrated over the course of several experiments to find an amount that satisfactorily inhibits the NFκB/IκB assemblage. Peptide agents that correspond to regions of NFκB or IκB that prevent the association of the NFκB/IκB complex will be detetected as a gel-retarded product comprising the radiolabeled oligonucleotide joined to NFκB, whereas peptide agents that fail to disrupt the NFκB/IκB complex will not be resolved by the gel retardation assay.

Additionally, the ability of the candidate peptide agents to inhibit IκB-mediated transcriptional repression in cells can be determined by treating cells that have been transfected with a NFκB reporter construct with varying concentrations of the peptide agents. A NFκB reporter construct can comprise, for example, three or more enhancer sequences (e.g., TGGGGATTCCCCA (SEQ. ID. NO. 1)) joined to a minimal promoter and a reporter molecule (e.g., luciferase, chloramphenicol acetyl transferase, or green fluorescent protein). Such a reporter construct can be made using conventional techniques in molecular biology. Preferably, the reporter construct is transfected into a cell line that has IκB and can produce copius amount of NFκB upon stimulation with a mitogen and a phorbal ester, such as Jurkat cells. Candidate peptide agents can be screened by transfecting the reporter construct in cells that have been cultured in the presence of varying concentrations of the peptide agents. By comparing the levels of reporter signal detected in untreated control cells to peptide agent-treated cells, the ability of a particular peptide agent to inhibit IκB-mediated transcriptional repression can be determined. Peptide agents that correspond to regions of NFκB or IκB that prevent the association of the NFκB/IκB complex will exhibit an increase in transcription in this assay, whereas peptide agents that fail to disrupt the NF-κB/IκB complex will have little if any transcription. In this manner, peptide agents that interupt the NFκB/IκB complex can be identified for incorporation into a pharmaceutical for the treatment and/or prevention of NFκB-related diseases.

In the disclosure below, the inventor discusses the manufacture, identification, and use of modified small peptides for the inhibition of bacterial toxin protein polymerization, which is necessary for the assembly of bacterial holotoxins.

The inhibition of toxicity of bacterial toxins Several bacterial toxins have supramolecular structures composed of polymerized proteins. For example, Bordetella Pertussis has a 105-kDa exotoxin, called pertussis toxin, that causes whooping cough, a highly contagious respiratory disease of infants and young children. Pertussis toxin consists of 5 polypeptide subunits (S1 to S5) arranged in an A-B structure typical of several bacterial toxins. (See, Read et al., U.S. Pat. No. 5,856,122). The S2, S3, S4 (two copies) and S5 subunits form a pentamer (the B oligomer) that when combined with the S 1 subunit forms the holotoxin. S1 is an enzyme with ADP-ribosyl transferase and NAD-glycohydrolase activities. SI activity is the primary cause of pertussis toxin (PT) toxicity.

The B oligomer mediates the binding of the holotoxin to target cells and facilitates entry of the A protomer. The function of this base structure is in binding to host cell receptors and enabling the S₁ subunit to penetrate the cytoplasmic membrane. (Armstrong and Peppler, Infection & Immun. 55:1294 (1987)). Pertussis toxin has been detoxified by modification of its cell binding properties, for example, by deletion of Asn-105 in the S2 subunit and Lys-105 in the S3 subunit, and by substitution of the Tyr-82 residue in S3. (Lobet et al., J Exp. Med. 177:79-87 (1993) and Loosmore et al., Infect. Immun. 61:2316-2324 (1993)). The 3-dimensional structure of pertussis toxin, as well as many other bacterial toxins, share functional and/or structural resemblance to PT, including diphtheria toxin, cholera toxin, Pseudomonas exotoxin A, the heat-labile toxin of E. coli, and verotoxin-1. (Read et al., U.S. Pat. No. 5,856,122, Choe et al., Nature 357:216-222 (1992), Allured et al., Proc. Natl. Acad. Sci. USA 83:1320-1324 (1986), Brandhuber et al., Proteins 3:146-154 (1988), Sixma et al., J. Mol. Biol. 230:8990-9180 (1993), Sixma et al., Biochemistry 32:191-198 (1993), and Stein et al., Nature 355:748-750 (1992)). This 3-dimensional information and the amino acid sequence that encodes the polypeptides of these bacterial toxins can be used to design and manufacture peptide agents that inhibit bacterial toxin subunit polymerization and, thus, the formation of bacterial toxin holotoxins.

By one approach, the 3-dimensional model of pertussis toxin is used to select protein-protein interacting regions that are susceptible to small peptide inhibition. One such region involves the interaction between the C-terminus of S1 (228 to 235) and the B-oligomer pore that accounts for 28% of the buried surface between S1 and the B-oligomer. Thus, one embodiment encompases peptide agents having sequence that corresponds to regions of S1 that interact with the B-oligomer (e.g., small peptides that correspond to overlapping sequences of S1 (228-235). Similarly, regions of S2, S3, S4, and S5 that compose the 28% of the buried surface between S1 and the B-oligomer are used to select and design peptide agents that inhibit the formation of the holotoxin.

Since dimerization of PT is of functional importance in binding to target cells, the interruption of this dimerization process by using peptide agents that correspond to regions of protein-protein interactions necessary for protein polymerization can provide a method to inactivate the holotoxin. Several residues in S2 contain unique amino acid determinants that promote dimerization. (Read et al., U.S. Pat. No. 5,856,122). The S2 residues Glu-66, Asp-81, Leu-82, and Lys-83, which are not conserved in S3, are predicted to be responsible for PT dimerization. Further, amino acid residues 82 and 83 are also important in glycoconjugate binding. Other regions of the S2 and S4 subunits, such as Trp-52 of S2 and residues Asp-1, Tyr-4, Thr-88, and Pro-93 of S4 are thought to be involved in protein-protein interactions that mediate polymerization of S2 and S4 subunits. Peptide agents that correspond to regions of the toxin subunits involved in assembly of the holotoxin are selected, designed, and manufactured. In a similar fashion, the selection, design, and manufacture of peptide agents that inhibit the polymerization of other bacterial toxin holoenzyrnes, such as diphtheria toxin, Pseudomonas exotoxin A, the heat-labile toxin of E. coli, and verotoxin-1, can be accomplished.

Next, the candidate peptide agents are screened in peptide characterization assays that evaluate their ability to bind to toxin subunit proteins, inhibit the formation of the holotoxin, and inhibit the toxic effects of the holotoxin. To evaluate the ability of a peptide agent to bind to PT holotoxin or individual proteins that compose the holotoxin, an in vitro binding assay is performed. As described earlier, there are several types of in vitro binding assays that are known in the art and a preferable approach involves the binding of radiolabeled peptide agents to PT proteins or holotoxin disposed in a dialysis membrane. By one approach, PT proteins or holotoxin are disposed in a dialysis membrane having a 10,000 mw cut-off (e.g., a Slide-A-lyzer, Pierce). Then, radioactively labeled peptide agents are added in a suitable buffer and the binding reaction is allowed to take place overnight at 4° C. The peptide agents can be radiolabeled with ¹²⁵I or ¹⁴C, according to standard techniques or can be labeled with other detectable signals. After the binding reaction has taken place, the peptide agent-containing buffer is removed, and the dialysis membrane having the protein of interest is dialyzed for two hours at 4° C. in a buffer lacking radioactive peptide agents. Subsequently, the radioactivity present in the dialyzed protein is measured by scintillation. Peptide agents that bind to PT proteins or holotoxin can be rapidly identified in this manner. Modifications of these binding assays can be employed, as would be apparent to those of skill in the art, in particular binding assays, such as described above are readily amenable to high throughput analysis, for example, by binding the PT proteins or holotoxin to a microtiter plate and screening for the binding of fluorescently labeled peptide agents.

After peptide agents that bind to PT proteins or holotoxin have been identified, assays that evaluate the ability of the peptide agents to disrupt the holotoxin are performed. Several of such assays are known in the art. Head et al. provide an approach that can be readily adapted to determine the ability of peptide agents to disrupt PT holotoxin into PT subunits. (Head et al., J. Biol. Chem. 266:3617 (1991)). Accordingly, in some experiments, purified PT (obtainable from List Biological Laboratories, Inc.) is incubated with peptide agents for 2 hrs at 4° C. In other experiments, purified PT is first dissociated in a dissociation buffer and then is brought back to a physiological buffer in the presence of a peptide agent, after which binding is allowed to occur for 2 h at 4° C. To bring the holotoxin to dissociating conditions, a dissociation buffer (6 M urea, 0.1 M NaCl, 0.1 M propionic acid, pH 4 is added dropwise, and the toxin is incubated without stirring at 4° C. for 1 h. (Ito et al., Microb. Pathog., 5, 189-195 (1988)). If the dissociation is performed in a small volume (e.g., 25 μl) and the dissociated subunits are resuspended in a large volume of physiological buffer containing a desired concentration of peptide agents (e.g., 975 μl), conditions that promote holotoxin formation and peptide agent binding can rapidly be restored. A suitable physiologic binding buffer is 50 mM Tris-buffered saline (TBS), pH 7.4.

After the binding reaction, holotoxin is resolved from dissociated complexes by high performance liquid chromatography (HPLC). Binding reactions containing approximately 1 mg of subunits or holotoxin (in 1 ml) are injected into a TSK-G2000SW HPLC gel filtration column previously equilibrated with 50 mM Tris-buffered saline (TBS), pH 7.4, flow rate of 1.0 ml/min. Peaks are then measured by absorbance at λ=280 nm, and fractions are collected. The purified PT will migrate as a single peak with a retention time of about 12-15 min. Dissociated subunits will present a profile having two peaks, representing the A subunit and B subunits. Peptide agents that disrupt the PT holotoxin or that prevent assembly of the holotoxin will be identified by the appearance of two peaks in the assay described above. Preferably, the concentration of the different peptide agents is titrated over the course of several experiments to find an amount that satisfactorily disrupts or prevents the assembly of the PT holotoxin.

Once peptide agents that disrupt or prevent the assembly of the PT holotoxin have been identified, the ability of such molecules to inhibit the toxic effects of PT are evaluated in a cell-based or animal based system. One cell-based assay analyzes the effects of PT on Chinese hamster ovary (CHO) cells in culture. The CHO cell assay is performed essentially as described by Hewlett et al. (Hewlett et al., Infect. Immun., 40: 1198 (1983)). CHO cells are grown and maintained in Ham F-12 (GIBCO Laboratories, Grand Island, N.Y.) medium containing 10% fetal calf serum and varying concentrations of the peptide agents in an atmosphere of 5% CO₂. Serial twofold dilutions of PT are prepared in Ham F-12 medium. Toxin is added in a volume of 10 μl to the CHO cells 20 h after they are put into the microtiter wells. After 24 h of additional incubation, the CHO cells are observed for the characteristic growth pattern associated with Toxin poisoning. That is, rounded, flat cells growing in tight clumps. In contrast, peptide agent treated cells (like the control cells, which were not administered toxin) will exhibit a monolayer of elongated cells.

By another approach, an animal-based study is performed to evaluate the ability of the peptide agents to interfere with the toxicity of PT. An animal based challenge to identify the efficacy of small peptides that correspond to sequence of pertussis toxin subunits can be employed as follows. Taconic mice (15 to 17 g) are injected at day zero with 0.5 ml of a modified small peptide intraperitoneally, in three doses so as to bring the concentration of the small peptide in the blood to 100 μM-300 μM. Each dose is injected into 10 mice. At day 2, the mice are challenged with an intracerebral injection of a standard dose of B. pertussis. Control mice are also injected at the same time to ascertain the effectiveness of the challenge. Three days after the challenge, the number of animal deaths is recorded every day up to and including day 28. At day 28, paralysed mice and mice with cerebral edema also are recorded as dead. Results are recorded as LD₅₀, which is the dose at which half the mice die. The result of this experiment will show that the LD₅₀ of small peptide treated mice is greater than untreated mice, and, thus, treatment with modified small peptides was protective against the disease. Peptide agents identified in this manner can be incorporated into pharmaceuticals for the treatment and prevention of the toxic effects of PT. Further, by using the approaches detailed above, peptide agents that disrupt or prevent assembly of other bacterial toxins, such as diphtheria toxin, Pseudomonas exotoxin A, the heat-labile toxin of E. coli, cholera toxin, and verotoxin-1 and 2 can be selected, designed, manufactured, and screened according to peptide characterization assays.

In other embodiments, disclosed below, modified small peptides are manufactured, identified, and used to inhibit the polymerization of proteins (e.g., actin and β-amyloid peptide) involved in the formation of supramolecular structures associated with the onset of nuerodegenerative diseases such as Alzheimer's disease and prion disease.

The Inhibition of Actin and β-amyloid Peptide Polymerization

Peptide agents can also be used to inhibit or prevent the polymerization of proteins that are involved in the onset of diseases associated with the aberrant assembly of fibrous proteins, such as Alzheimer's disease (AD) and prion disease. Like AD, the human prion diseases, Creutzfeldt-Jakob disease and Gertsmann-Sträussler-Scheinker disease, are characterized by the slow onset of neurodegeneration. Brain pathology in these diseases resembles that of AD and is also characterized by aggregation of a normal cellular protein, prion protein (PrP) (rather than the β-amyloid peptide associated with AD). (Baker and Ridley, Neurodegeneration, 1: 3-16 (1992), (Prusiner, N. Engl. J Med. 310: 661-663(1984), and (Prusiner, Science 252: 1515-1522 (1991)).

The infective agent of scrapie is believed to operate by accelerating the step in amyloid formation that is normally rate determining. (Griffith, Nature 215: 1043-1044 (1967) and (Prusiner, Science 252: 1515-1522 (1991)). Many believe that this step—the formation of an ordered nucleus, which is the defining characteristic of a nucleation-dependant polymerization—is mechanistically relevant to amyloid formation in human prion disease and in AD. (Jarret and Lansbury Cell, 73:1055-1058 (1993)). Thus, a disruption of the seeding of amyloid formation can be an approach to treat or prevent the transmission of scrapie and the initiation of AD.

Nucleation-dependent protein polymerization describes may well-characterized processes, including protein crystallization, microtubule assembly, flagellum assembly, sickle-cell hemoglobin fibril formation, bacteriophage procapsid assembly, and actin polymerization. By one interpretation, nucleus formation requires a series of association steps that are thermodynamically unfavorable (Kₙ<<1) because the resultant intermolecular interactions do not outweigh the entropic cost of association. (Chothia and Janin, Nature, 256: 705 (1975)). Once the nucleus has formed, further addition of monomers becomes thermodynamically favorable (K_{g}>>1) because monomers contact the growing polymer at multiple sites, resulting in rapid polymerization/growth. That is, nucleation is rate determining at low supersaturation levels. Therefore, adding a seed or preformed nucleus to a kinetically soluble supersaturated solution results in immediate polymerization. However, by determining the regions of the seed that are necessary for the protein-protein interactions that enable polymerization, peptide agents can be selected and designed to these regions and identified according to their ability inhibit or prevent “seeding” or polymerization. Such peptide agents can be incorporated into pharmaceuticals and can be administered for the treatment and prevention of nuerodegenerative diseases like AD and prion disease. The use of β-amyloid peptides having 6-60 amino acid residues joined to modulating group such as biotin and other cyclic and heterocyclic compounds and other compounds having similar steric “bulk” have been reported to inhibit aggregation of natural β-amyloid peptides. (U.S. Pat. No. 5,817,626).

Pathologically, Alzheimer's disease (AD) is characterized by the presence of distinctive lesions in the victim's brain. These brain lesions include abnormal intracellular filaments called nuerofibrillary tangles (NFTs) and extracellular deposits of amyloidogenic proteins in senile, or amyloid, plaques. The major protein constituent of amyloid plaques has been identified as a 4 kilodalton peptide (40-42 amino acids) called β-amyloid peptide. (Glenner et al., Biochem. Biophys. Res. Commun. 120:885-890 (1984) and Masters et al., Proc. Natl. Acad. Sci. USA 82:4245-4249 (1985)). Diffuse deposits of β-amyloid peptide are frequently observed in normal adult brains, whereas AD brain tissue is characterized by more compacted, dense-core β-amyloid plaques. (See, e.g., Davies et al., Neurology 38:1688-1693 (1988)). The neurotoxicity of β-amyloid peptide is dependent upon its ability to “seed” aggregates or polymers that accumulate at plasma membranes and disrupt cellular calcium homeostasis. Calcium influx through glutamate receptors and voltage dependent channels mediates an array of function and structural responses in neurons. Unrestrained calcium influx, however, can injure and kill neuronal cells. Aggregation or polymerization of β-amyloid peptides can cause a drastic influx of calcium, which injures or kills nerve cells.

Actin microfilaments are a major cytoskeletal element whose polymerization state is highly sensitive to calcium. Cytochalasin compounds cause actin depolymerization, reduce calcium influx induced by glutamate and membrane depolarization, and abrogate the calcium influx mediated by β-amyloid polymerization at plasma membranes. (Mattson, U.S. Pat. No. 5,830,910). Thus, the actin microfilaments that compose the cytoskeleton play an active role in modulating calcium homeostasis and compounds that affect actin polymerization can alleviate neuronal injury in a variety of neurodegenerative conditions. Thus in other embodiments, peptide agents that correspond to sequences of actin involved in actin polymerization are selected, designed, manufactured, and identified according to their ability to inhibit actin polymerization and, thereby, counteract the calcium influx induced by β-amyloid peptide aggregation. Similarly, peptide agents that correspond to sequences of β-amyloid peptide can be used to prevent aggregation of β-amyloid peptide at plasma membranes and, thereby, counteract the calcium influx induced by β-amyloid peptide aggregation. Further, therapies that combine peptide agents that correspond to regions of actin and β-amyloid protein are within the scope of some embodiments of the invention.

Peptide agents that correspond to actin and β-amyloid peptide sequences involved in polymerization can be designed, manufactured, and identified by employing the strategy described above. Again, generally, mutation analysis, protein modeling and drug interaction analysis in the literature is reviewed or such determinations are made by conventional approaches to design and select appropriate peptide agents that correspond to sequences involved in protein polymerization. Of course, small peptides can be selected at random. The peptide agents are then manufactured (e.g., by using the approach detailed above). Next, the selected small peptides are identified by conducting peptide characterization assays that evaluate the ability of the peptide agent to bind to a protein of interest, inhibit or prevent polymerization or binding of the protein, and reduce a disease state associated with the polymerized protein or supramolecular assembly. Any number or order of peptide characterization assays can be employed to identify a small peptide that inhibits protein polymerization or supramolecular complex assembly.

Since cytochalasins bind to the rapidly growing (barbed) end of actin and, thereby, block all association and disassociation reactions, small peptides corresponding to actin sequences at the barbed end will interfere with actin polymerization. Thus, peptide agents that correspond to this region of actin are selected, designed, and manufactured.

The mutation and substitution of two hydrophobic amino acids of β-amyloid peptide has been shown to reduce amyloidogenicity. (Hilbich et al., J. Mol. Biol. 228:460-473 (1992)). A well-preserved hydrophobic core around residues 17 to 20 of β-amyloid peptide was found to be important for the formation of P-sheet structures and other amyloid properties. This region is believed to play an important role in assembling and stabilizing amyloid plaques. Thus, peptide agents that correspond to this region of β-amyloid peptide are selected, designed, and manufactured.

Once made, the peptides are screened in peptide characterization assays. To evaluate the ability of a peptide agent to bind to actin or β-amyloid peptide (purified forms are obtainable from Sigma), an in vitro binding assay is performed with radiolabeled peptide agents. As described previously, a preferred approach involves disposing the protein of interest in a dialysis membrane and binding the protein with radiolabeled peptide agents. Accordingly the protein of interest is placed in a dialysis membrane having a 10,000 mw cut-off (e.g., a Slide-A-lyzer, Pierce). Then, radioactively labeled peptide agents are added in a suitable buffer and the binding reaction is allowed to take place overnight at 4° C. The peptide agents can be radiolabeled with ¹²⁵I or ¹⁴C, according to standard techniques or can be labeled with other detectable signals. After the binding reaction has taken place, the peptide agent-containing buffer is removed, and the dialysis membrane having the protein of interest is dialyzed for two hours at 4° C. in a buffer lacking radioactive peptide agents. Subsequently, the radioactivity present in the dialyzed protein is measured by scintillation. Peptide agents that bind to the actin or β-amyloid peptide can be rapidly identified in this manner. Modifications of these binding assays can be employed, as would be apparent to those of skill in the art, in particular binding assays, such as described above are readily amenable to high throughput analysis, for example, by binding the actin or β-amyloid peptide to a microtiter plate and screening for the binding of fluorescently labeled peptide agents.

After peptide agents that bind to actin or β-amyloid peptide have been identified, assays that evaluate the ability of the peptide agents to disrupt polymerization of actin or β-amyloid peptide are performed. In so far as the inhibition of actin polymerization is concerned, techniques in immunohistochemistry can be used. Accordingly, immunofluorescence studies are conducted on cells that have been treated with peptide agents and the presence of polymerized actin is determined with antibodies that are specific for actin (e.g., Monoclonal anti-actin-FITC conjugate (Clone No. AC-40) Sigma F3046). Transformed mouse neuroblastoma cells and normal fibroblast cells are suitable for these experiments and such cells are contacted with varying amounts of peptide agents, fixed, stained with the anti-actin antibody, and are analyzed according to standard immunofluorescence techniques.

By one approach, cells of transformed mouse neuroblastoma clone N1E-1 15 are grown in Dulbecco's modified Eagles median (DMEM) supplemented with 5% fetal calf serum at 37° C. in an atmosphere of 10% CO₂. Normal mouse fibroblasts (Swiss/3T3) are grown in DMEM supplemented with 10% fetal calf serum. The cells are contacted with 100 μM-300 μM of peptide agents overnight or no peptide agents (control) and are subsequently re-plated onto 35-mm plastic tissue culture dishes containing glass cover slips. Differentiated neuroblastoma cells are obtained by adding 2% dimethyl sulfoxide (DMSO) to the growth medium.

The cells on the cover slip are then cooled on ice, the culture media is removed, and the cells are washed in cold phosphate-buffered saline (PBS). After washing, the cells are fixed for 30 minutes in 2% paraformaldehyde (PFA), a 1:1 dilution with PBS of 4% PFA, and 0.1% Triton X-100 on ice, or 15 minutes in 100% methanol at −10° C. After fixation, the fixative is removed and the cells are washed twice in 4° C. PBS (5 minutes/wash). The FITC labeled anti-actin antibody is added at a 1:75 dilution and binding is allowed to take place for 1 hour at 4° C. Subsequently, the cells are washed four times in 4° C. PBS (5 minutes/wash).

Microscopic examination of the cells will reveal that untreated cells have extensive actin microfilaments labeled with the FITC anti-actin antibody. Untreated cells will show organized actin characterized by long actin bundles. The neuroblastoma cells, in particular, will show a smooth contour, typified by microspikes. In constrast, cells treated with the peptide agents that correspond to sequences of actin that are involved in actin polymerization, will show rounded up cells, a loss of microspikes and altered growth cones. Additionally, the long actin bundles found in normal cells will no longer be visible and intense labeling of actin will be found in the cytoplasm or in the ruffling membranes. By using the techniques described above, peptide agents that correspond to actin protein sequence can be designed, manufactured, and screened for the ability to bind to actin and prevent actin polymerization. As an added positive control, cells can be treated with a cytochalasin compound and immunofluouresence will show a depolymerization of actin characterized by the lack of long actin bundles.

Regarding the determination of agents that inhibit β-amyloid peptide aggregation/polymerization, several methods are known. By one approach, β-amyloid protein₍₁₋₄₀₎ is dissolved in hexafluoro isopropynol (HFIP; Aldrich Chemical Co) at 2 mg/ml. Aliquots of the HFIP solution are transferred to test tubes and a stream of argon gas is passed through each tube to evaporate the HFIP. The resulting thin film of β-amyloid peptide is dissolved in DMSO and a small teflon-coated magnetic stir bar is added to each tube. A suitable buffer (e.g., 100 mM NaCl, 10 mM sodium phosphate pH 7.4) is added to the DMSO solution with stirring. The resulting mixture is stirred continuously and the optical density is monitored at 400 nm to observe the formation of insoluable peptide aggregates. In control samples, peptide aggregates will be readily discernible as determined by an increase in optical density at 400 nm. In the presence of peptide agents, however, β-amyloid peptide aggregation will be inhibited as detected by a lower optical density at 400 nm than the control sample.

In a second assay, β-amyloid protein aggregation is measured using a fluorometric assay. (Levine, Protein Science 2:404-410 (1993)). In this assay, the dye thioflavine T (ThT) is contacted with the β-amyloid protein solution. The dye ThT associates with aggregated β-amyloid protein but not monomeric or loosely associated β-amyloid protein. When associated with β-amyloid protein, ThT gives rise to a excitation maximum at 450 nm and an enhanced emission at 482 nm compared to the 385 nm and 455 nm for the free dye. Accordingly, aliquots of β-amyloid protein in the presence and absence of peptide agents that correspond to sequences of β-amyloid protein, are added to reaction vessels and brought to 50 mM potassium phosphate buffer pH 7.0 containing thioflavin T (10 mM; obtained from Aldrich Chemical Co.). Excitation is set at 450 nm and emission is measured at 482 nm. As in the aggregation assay above, samples that have peptide agents that inhibit aggregation of β-amyloid peptide will show little emission at 482 nm as compared to 444 nm, the emission for the free dye, whereas, control samples will show considerable emission at 482 nm and little emmission at 444 nm.

In a third assay, the ability of peptide agents of the invention to disrupt β-amyloid aggregation is determined by mixing the β-amyloid peptides with peptide agents and staining the mix with Congo red. All types of amyloid show a green birefringence under polarized light if they are stained with the dye Congo red. However, β-amyloid peptides that are unable to aggregate by virtue of the presence of peptide agents will not exhibit a green birefringence under polarized light. Accordingly, approximately 0.5 to 1 mg of freeze-dried β-amyloid peptides are suspended in 100 μl of PBS, pH 7.4 containing 100 to 300 μM peptide agent. After the addition of the β-amyloid peptides, 5 μl of a Congo red solution (1% in water) is added. Then 20 μl of the suspension is placed onto a microscope slide and inspected immediately under polarized and non-polarized light in a microscope. Photographs can be taken at a primary magnification of 200×. In control samples, e.g., no peptide agents, aggregated β-amyloid peptides and a green birefringence will be observed, however, samples having peptide agents will show reduced β-amyloid aggregation and green birefringence.

Additionally, β-amyloid aggregation in the presence and absence of peptide agents can be assessed by using electron microscopy. For filament formation, solutions of β-amyloid peptides in 70% HCOOH (1 mg β-amyloid peptide/200 μl ) are dialysed against a mixture of PBS and HCOOH with and without peptide agents at room temperature for 5 days. During this time the amount of PBS in the dialysis buffer is increased from 20 to 100%. Fresh suspensions of β-amyloid peptides in PBS with and without peptide agents (after dialysis) are applied to carbon-coated, deionized copper grids, dried, negatively stained with 2% (w/v) uranyl acetate and are visualized in an electron microscope. A characteristic feature of β-amyloid peptides is their tendency to aggregate into insoluble filaments of great molecular mass. Such aggregates are readily detected by electron microscopy and can have a diameter of about 5 nm with a length that approaches 200 nm. Samples containing β-amyloid peptides that were contacted with peptide agents, however, will show few if any filaments.

To ascertain the ability of peptide agents that correspond to actin sequence and β-amyloid sequence to disrupt the calcium influx induced by β-amyloid peptide aggregation, functional assays using hippocampal cell cultures are performed. Disassociated embryonic rat hippocampal cell cultures are established and maintained on a polyethyleneimine-coated substrate in plastic 35-mm dishes, 96 well plates, or glass-bottom 35-mm dishes. The cell density is maintained at approximately 70-100 cells/mm. The cells are maintained in Eagles minimum essential medium supplemented with 10% fetal bovine serum containing 20 mM sodium pyruvate. The experiments are performed on 6-10 day-old cultures, a time at which neurons exhibit calcium responses to glutamate mediated by both NMDA and oc-amino-3-hydroxy-5-methylisoxazole-4-propionic acid (AMPA)/kainate receptors, and are vulnerable to excitotoxicity and β-amyloid toxicity. β-amyloid peptide 25-35 and 1-40 (Sigma A1075, A4559, respectively) are prepared immediately before use by dissolving the peptide at a concentration of 1 mM in sterile distilled water. These peptides aggregate rapidly when placed in culture medium and will progressively kill neurons over a 48-hour period when added to cultures in a soluble form. (Mattson, U.S. Pat. No. 5,830,910, herein incorporated by reference).

Neuronal survival is quantified by counting viable neurons in the same microscopic field (10× objective) immediately before treatment and at time points after treatment. Additionally, cells grown in 96-well plates in the presence of Alamar blue fluourecense (Alamar Laboratories) is quantified by using a fluourescense plate reader. Alamar blue is a non-fluourescent substrate that after reduction by cell metabolites, becomes fluourencent. Viability of neurons is assessed by morphological criteria. Neurons with intake neurites of uniform diameter and a soma with a smooth, round appearance are considered viable, whereas neurons with fragmented neurites and a vacuolated or swollen soma are considered non-viable.

Survival values can be expressed as percentages of the initial number of neurons present before experimental treatment. In the presence of peptide agents that correspond to actin sequences and/or β-amyloid sequences that are necessary for protein polymerization, a greater than 50% neuron survival will be observed. Desirably, neuron survival induced by contacting the cells with a peptide agent that corresponds to an actin or β-amyloid peptide sequence or both sequences will be between 50-100%. Preferably, neuron survival will be 60-100% and neuron survival can be 55%, 60%, 65%, 70%, 75%, 80%, 85%, 90%, 95% and 100%. In contrast, cells incubated with 100 mM glutamate will show a less than 25% neuron survival and cells cultured in the presence of β-amyloid peptides will show a neuron survival of less than 50%. Further, in cells pretreated for 1 hour with the peptide agents that correspond to actin sequences and/or β-amyloid peptides, glutamate neurotoxicity will be reduced.

In further studies, a measurement of calcium influx in the presence and absence of peptide agents that correspond to actin and/or β-amyloid peptide sequences can be determined by using the calcium indicator dye Fura-2. By one approach, fluoresence ratio imaging of the Ca²+indicator dye Fura-2 is used to quantify Ca²+in neuronal somata that has been treated with either glutamate or β-amyloid peptide in the presence and absence of peptide agents that correspond to either actin or β-amyloid peptide sequences or both. Cells are incubated for 30-40 minutes in the presence of 2 mM acetoxymethyl ester form of the Ca²+indicator dye Fura-2 and are then washed twice (2 ml/wash) with fresh medium and are allowed to incubate at least 40 minutes before imaging. Immediately before imaging, normal culture medium is replaced with Hanks balanced saline solution (Gibco) containing 10 mM HEPES buffer and 10 mM glucose. Cells are imaged using a Zeiss Attofluor system with an oil objective or Quantex system with a 40× oil objective. However, those of skill in the art will appreciate that other microscopic systems can be employed.

The ratio of fluoresence emission using two different excitation wave lengths (334 and 380 nm) is used to determined calcium influx. The system is calibrated using solutions containing either no Ca²⁺ or a saturating of Ca²⁺ (1 mM). Fura-2 calcium imaging will reveal that peptide agents that correspond to sequences of actin or amyloid peptide or both will attenuate [Ca²⁺]ᵢ responses to glutamate and β-amyloid peptide induced membrane depolarization. In control cultures, for example, 50 mM glutamate will induce a rapid increase in neuronal [Ca²⁺]ᵢ. In contrast, [Ca²⁺]ᵢ response to glutamate in neurons pretreated with 300 μM peptide agents for one hour is reduced. Additionally, the neuronal [Ca²⁺]ᵢ response to glutamate is greatly enhanced in cultures pretreated with β-amyloid peptides for 3 hours. However, in the presence of peptide agents corresponding to actin or β-amyloid peptide sequences, the potentiation of [Ca²⁺]ᵢ response to glutamate in β-amyloid-pretreated culture is suppressed. These experiments will demonstrate that actin depolymerization and or β-amyloid peptide depolymerization caused by the presence of peptide agents corresponding to sequences of actin and β-amyloid peptide will reduce [Ca²⁺]ᵢ influx induced by glutamate and β-amyloid mediated membrane depolarization.

As mentioned in the foregoing section, a combination therapy employing both peptide agents that correspond to actin sequence and β-amyloid peptide sequence are embodiments of the invention. By using the assays described above, peptide agents that bind to actin and β-amyloid peptide can be selected, designed, manufactured and characterized. A better response (e.g., less Ca²⁺ influx) can be obtained by administering peptide agents that correspond to sequences of both actin and β-amyloid peptide. Additionally, by using approaches similar to those described above, peptide agents that inhibit the formation of prion-related protein plaques can be selected, designed, manufactured and characterized. Peptide agents selected, designed, manufactured and characterized as described above can be incorporated into pharmaceuticals for use as therapeutic and prophylactic agents for the treatment and prevention of nuerodegenerative diseases such as Alzheimer's disease and prion disease. Methods of treatment of subjects afflicted with nuerodegenerative orders such as Alzheimer's disease are performed by administering such pharmaceuticals. (See Findeis et al., U.S. Pat. No. 5,817,626 for modulators of β-amyloid peptide aggregation). Further, the efficacy of such peptides can be tested in transgenic mice that exhibit an Alzheimer-type neuropathology. (Gains et al., Nature 373:523-527 (1995)). These transgenic mice express high levels of human mutant amyloid precursor protein and progressively develop many of the pathological conditions associated with Alzheimer's disease. In the disclosure below, use of the PPI technology to interrupt tubulin polymerization for the treatment and prevention of cancer is described.

Inhibition of Tubulin Polymerization

In another aspect, the manufacture and use of peptide agents for the inhibition of tubulin polymerization is described. The peptide agents that inhibit tubulin polymerization are used as biotechnological tools and as therapeutics for the treatment of various forms of cancer. Peptide agents that correspond to sequences of tubulin α or β subunits or both, for example, can prevent tubulin polymerization and can be used as anti-tumor agents. The small peptide-tubulin polymerization inhibitors can be incorporated into pharmaceuticals for treating leukemias, melanomas and colon, lung, ovarian, CNS, and renal cancers, as well as other cancers. Preferably, the peptide agents are used to treat colon cancers.

A variety of clinically-promising compounds that demonstrate potent cytotoxic and anti-tumor activity are known to effect their primary mode of action through an efficient inhibition of tubulin polymerization. (Gerwick et al., J. Org. Chem. 59:1243 (1994)). This class of anti-tumor compounds binds to tubulin and in turn arrests the ability of tubulin to polymerization into microtubules which are essential compounds for cell maintenance and cell division. (Owellen et al., Cancer Res. 36:1499 (1976)). Currently, the most recognized and clinically useful tubulin polymerization inhibitors for the treatment of cancer include vinblastine, vincristine, rhizoxin, combretastin A-4 and A-2, and taxol. (Pinney, U.S. Pat. No. 5,886,025).

Tubulin is a heterodimer of globular α and β tubulin subunits. By using photoaffinity labeling reagents for tubulin, investigators have identified three distinct small molecule binding sites on tubulin: the colchicine site, the vinblastine site, and the rhizoxin site. Additionally, photoaffinity labeling reagents have revealed that rhizoxin binds to Met-363-Lys-379 site on β-tubulin. (Sawada et al., Biochem. Pharmacol. 45:1387 (1993)). Further, a taxol-based reagent has been found to label the N-terminal 31 amino acid residues of β-tubulin. (Swindell et al., J. Med. Chem. 37:1446 (1994) and Rao et al., J. Biol. Chem. 269:3132 (1994)). Preferably, the peptide agents of these embodiments are selected and designed to correspond to sequences in these regions.

Once selected, designed, and manufactured, the peptide agents are screened for their ability to bind to tubulin. By using an approach similar to that described above, tubulin (Sigma T 4925) is placed is a dialysis membrane, (e.g., a Slide-A-lyzer, Pierce). Then, radioactively labeled peptide agents are added in a suitable buffer and the binding reaction is allowed to take place overnight at 4° C. The peptide agents can be radiolabeled with ¹²⁵I or ¹⁴C, according to standard techniques or can be labeled with other detectable signals. After the binding reaction has taken place, the peptide agent-containing buffer is removed, and the dialysis membrane having the protein of interest is dialyzed for two hours at 4° C. in a buffer lacking radioactive peptide agents. Subsequently, the radioactivity present in the dialyzed protein is measured by scintillation. Peptide agents that bind to the tubulin are rapidly identified by the detection of radioactivity in the scintillation fluid. Modifications of these binding assays can be employed, as would be apparent to those of skill in the art, in particular binding assays, such as described above are readily amenable to high throughput analysis, for example, by binding the tubulin to a microtiter plate and screening for the binding of fluorescently labeled peptide agents.

After peptide agents that bind to tubulin have been identified, assays that evaluate the ability of the peptide agents to disrupt tubulin polymerization are performed. One suitable assay system is that described by Bai et al., Cancer Res. 56:4398-4406 (1996). inhibition of glutamate-induced assembly of purified tubulin in the presence and absence of peptide agents can be evaluated in 0.25-ml reaction mixtures following preincubation for 15 min at 37° C. without GTP. Final concentrations for a typical reaction mixture can be 1.0 mg/ml (10 μM) tubulin, 300 μM peptide agent, 1.0 M monosodium glutamate, 1.0 mM MgCl₂, 0.4 mM GTP, and 4% (v/v) DMSO. Assembly is initiated by a 75-s-jump from 0 to 37° C. and can be monitored in a Gilford spectrophotometer at 350 nm. The extent of the reaction is evaluated after 20 min. In the presence of peptide agents, very little absorbance at 350 nm will be detected. In contrast, in the absence of peptide agents, significant absorbance at 350 nm will be detected.

Tubulin aggregation in the presence and absence of peptide agents can also be followed by HPLC on a 7.5×300 -mm TSK G3000SW gel permeation column with an LKB system in line with a Ramona 5-LS flow detector. The column is equilibrated with a solution containing 0.1 M MES (pH 6.9) and 0.5 mM MgCl₂. Absorbance data can be evaluated with Raytest software on an IBM-compatible computer. In the presence of peptide agents, very little absorbance at 350 nm will be detected. In contrast, in the absence of peptide agents, significant absorbance at 350 nm will be detected. Further, electron microscopy can be used to evaluate tubulin aggregation in the presence and absence of peptide agents. Accordingly, 5 μl of the reaction is placed on a 200-mesh, carbon-coated, Formavar-treated copper grid, and after 5-10 s, the unbound sample is washed off with 5-10 drops of 0.5% uranyl acetate. Excess stain is removed by absorbance into torn filter paper and the negatively stained specimens are examined in an electron microscope. In the presence of peptide agents, very few tubulin bundles will be seen. In contrast, in the absence of peptide agents, a significant number of tubulin bundles will be observed.

The peptide agents can also be tested for their ability to inhibit tumor cell growth. The cytotoxicity of peptide agents that correspond to sequences of tubulin are evaluated in terms of growth inhibitory activity against several human cancer cell lines, including ovarian CNS, renal, lung, colon and melanoma lines. The assay used is described in Monks et al. (See e.g., Monks et al., J. Nat. Cancer Inst., 83:757-766 (1991), herein incorporated by reference). Briefly, cell suspensions, diluted according to the particular cell type and the expected target cell density (approximately 5,000-40,000 cells per well based on cell growth characteristics), are added by pipet (100 μ.l) to 96-well microtiter plates. Inoculates are allowed a preincubation time of 24-28 hours at 37° C. for stabilization. Incubation with the peptide agents is allowed to occur for 48 hours in 5% CO₂ atmosphere and 100% humidity.

Determination of cell growth is accomplished by in situ fixation of cells, followed by staining with a protein-binding dye, sulforhodamine B (SRB), which binds to the basic amino acids of cellular macromolecules. The solubilized stain is measured spectrophotometrically. The peptide agents that correspond to sequences of tubulin are preferably evaluated for cytotoxic activity against P388 leukemia cells. The ED₅₀ value, defined as the effective dosage required to inhibit 50% of cell growth) can be determined for each of the peptide agents tested. Cancer cells incubated in the presence of peptide agents will exhibit very little proliferation and cell growth, whereas, in the absence of peptide agents, the cancer cells will proliferate. Peptide agents selected, designed, manufactured and characterized as described above can be incorporated into pharmaceuticals for use as therapeutic and prophylactic agents for the treatment and prevention of various forms of cancer. The disclosure below discusses the use of PPI technology to disrupt viral capsid assembly for the treatment and prevention of viral infection.

Inhibition of Viral Capsid Assembly

Another aspect includes the manufacture and use of peptide agents for the inhibition of viral infection. The peptide agents that inhibit viral infection are used as biotechnological tools and as therapeutics for the treatment of various forms of viral disease. Peptide agents that correspond to sequences of the viral capsid protein, for example, can prevent polymerization of the capsid and can be used as an anti-viral agent. These anti-viral peptide agents can be incorporated into pharmaceuticals for treating HIV-1, HIV-2, and SIV, as well as, types of viral infections.

Initially, peptide agents that correspond to the viral capsid protein of HIV-1, HIV-2, and SIV (“p24”) were selected, designed and manufactured. The p24 protein polymerizes to form the viral capsid and is an integral component for the formation of the lentivirus nucleocapsid. The amide form of the small peptides listed in Table 1, which correspond to sequences of p24 believed to be involved in the protein-protein interactions that enable polymerization of the capsid, were manufactured and screened in characterization assays. These peptide agents were synthesized according to the method disclosed earler, but could of course be synthesized by any method known in the art.

TABLE 1

| | |
|---|---|
| Leu-Lys-Ala (LKA) | Arg-Gln-Gly (RQG) |
| Iso-Leu-Lys (ILK) | Lys-Gln-Gly (KQG) |
| Gly-Pro-Gln (GPQ) | Ala-Leu-Gly (ALG) |
| Gly-His-Lys (GHK) | Gly-Val-Gly (GVG) |
| Gly-Lys-Gly (GKG) | Val-Gly-Gly (VGG) |
| Ala-Cys-Gln (ACQ) | Ala-Ser-Gly (ASG) |
| Cys-Gln-Gly (CQG) | Ser-Leu-Gly (SLG) |
| Ala-Arg-Val (ARV) | Ser-Pro-Thr (SPT) |
| Lys-Ala-Arg (KAR) | Gly-Ala-Thr (GAT) |
| His-Lys-Ala (HKA) | Lys-Ala-Leu (KAL) |
| Gly-Pro-Gly (GPG) | |

Abbreviations Used:
Leu-Leucine
Gln-Glutamine
His-Histidine
Cys-Cysteine
Pro-Proline
Val-Valine
Ser-Serine
Lys-Lysine
Ala-Alanine
Ileu-Isoleucine
Gly-Glycine
Arg-Arginine
Thr-Threonine

To determine whether the peptide agents listed in Table 1 bound to the viral capsid protein p24, an in vitro binding assay was performed. As described previously, a dialysis-based binding assay was conducted using a dialysis membrane with a pore size of less than 10 kD. (Slide-A-Lyzer, Pierce). Fifty microliters of a 10 μM stock of the recombinant proteins p24, gp120 (gifts from the AIDS program, NCIB) and BSA (Sigma) were introduced into separate dialysis membranes and the proteins were dialyzed at 4° C. for 2 days against a 500 ml solution composed of 150 mM NaCl and 50 mM Tris-HCl, pH 7.4 buffer, and 27.5 μM of ¹⁴C-GPG-NH₂ (Amersham Ltd. UK). Subsequently, ten or five microliter aliquots of the dialyzed p24, gp 120, and BSA were removed and mixed with 3 ml of ReadySafe (Beckman) in a scintillation vial. The C¹⁴ was then detected by scintillation counting.

In Table 2, the results from a representative dialysis-based binding assay are provided. Notably, an association of p24 with GPG-NH₂ was observed upon dialysis equilibration. The amount of radioactive GPG-NH₂ associated with p24 was 7.5 times greater than that present in the buffer. In contrast, no appreciable amount of radioactive GPG-NH₂, over the amount present in the dialysis buffer, was associated with either gpl20 or BSA. These results prove that small peptides, such as GPG-NH₂, bind to p24.

TABLE 2

| Sample: | dialysis buffer | p24 | gp120 | BSA |
|---|---|---|---|---|
| μCi/ml | 1.816 | 13.712 | 1.745 | 1.674 |
| times buffer | 1.000 | 7.551 | 0.961 | 0.922 |

Evidence that peptide agents inhibit or prevent viral capsid protein polymerization and, thus, proper nucleocapsid assembly was obtained by performing electron microscopy on HIV-1 particles that were contacted with a modified small peptide. In this set of experiments, HUT78 cells were infected with HIV-1 SF-2 virus at 300TCID₅₀ for 1 hr at 37° C. Subsequently, the infected cells were washed and pelleted 3 times. Thereafter, the cells were resuspended in RPMI culture medium supplemented with 10% FBS, antibiotics (100 u/ml) and polybrene (3.2 μg/ml). GPG-NH₂ was then added into the cell cultures 3, 5 or 7 days post infection at concentration of 1 μm or 10 μM. A control sample was administered 0.5 μM Ritonavir (a protease inhibitor). The cells were cultured until day 14, at which point, the cells were fixed in 2.5% glutaraldehyde by conventional means. The fixed cells were then post-fixed in 1% OSO₄ and were dehydrated, embedded with epoxy resins, and the blocks were allowed to polymerize. Epon sections of virus infected cells were made approximately 60-80 nm thin in order to accommodate the width of the nucleocapsid. The sections were mounted to grids stained with 1.0% uranyl acetate and were analyzed in a Zeiss CEM 902 microscope at an accelerating voltage of 80 kV. The microscope was equipped with a spectrometer to improve image quality and a liquid nitrogen cooling trap was used to reduce beam damage. The grids having sections of control and GPG-NH₂ incubated cells were examined in several blind studies.

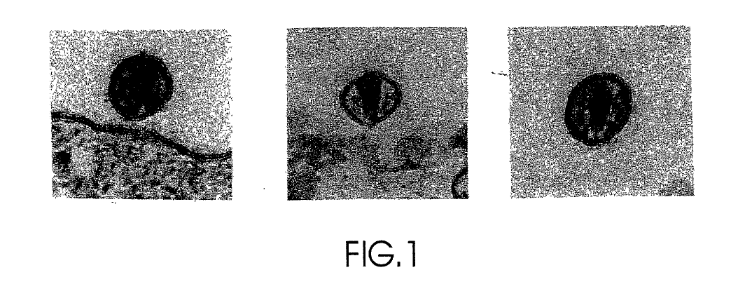

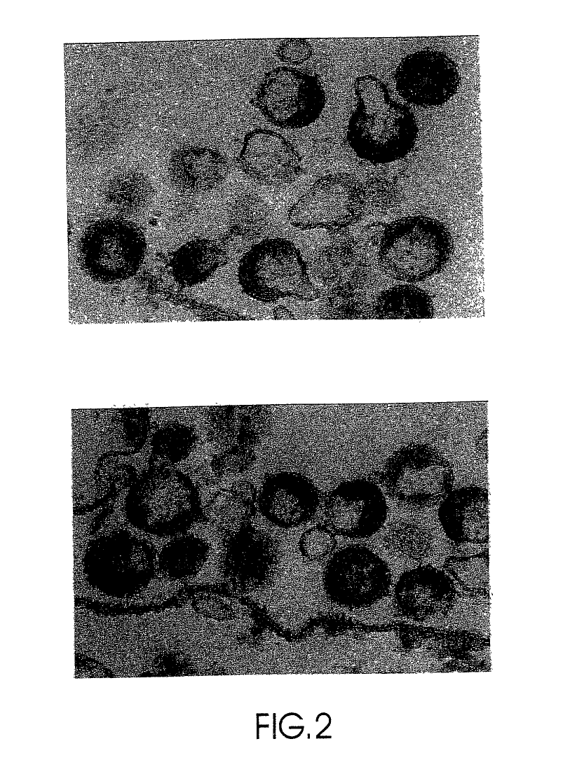

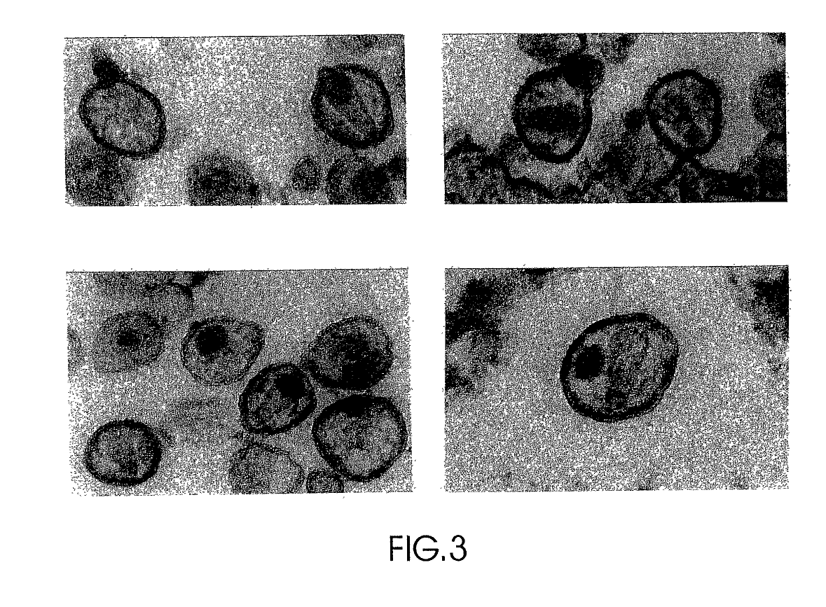

Electron microscopy of untreated HIV particles revealed the characteristic conical-shaped nucleocapsid and enclosed uniformly stained RNA that stretched the length of the nucleocapsid. (See FIG. 1). In contrast, FIG. 2 presents two electron micrographs showing several HIV-1 particles that have been contacted with the viral protease inhibitor Ritonavir. Infected cells that had been treated with Ritonavir exhibited malformed structures that did not have a discernable nucleocapsid, as was expected. FIG. 3 presents electron micrographs showing viral particles that had been contacted GPG-NH₂. Cells having HIV-1 particles that were contacted with GPG-NH₂ exhibited HIV-1 particles with discernable capsid structures that are distinct from the Ritonavir-treated particles. More specifically, in some tripeptide-treated viral particles, the conical-shaped capsid structure appeared to be partially intact but the RNA was amassed in a ball-like configuration either outside the capsid or at the top (wide-end) of the capsid. Still further, some capsids were observed to have misshapen structures with little or no morphology resembling a normal nucleocapsid and RNA was seen to be either outside the structure or inside the structure at one end. From these studies it was clear that small peptides interfered with viral capsid protein polymerization and proper formation of the nucleocapsid.

Next, the ability of peptide agents to inhibit viral infection was evaluated. Accordingly, the peptide agents listed in Table 1 were used in several viral (e.g., HIV-1, HIV-2, and SIV) infection assays. The efficiency of HIV-1, HIV-2, and SIV infection was monitored by reverse transcriptase activity, the concentration of p24 protein in the cell supernatent, and by microscopic evaluation of HIV-1 syncytia formation. In initial experiments, several modified tripeptides were screened for the ability to inhibit HIV-1, HIV-2, and SIV infection in H9 cells. Once inhibitory tripeptides were identified, more specific assays were conducted to determine the effect of varying concentrations of the selected tripeptides and combination treatments (e.g., the use of more than one modified tripeptide in combination).

In Experiments 1 and 2, approximately 200,000H9 cells were infected with HIV-1, HIV-2 or SIV at 25 TCID₅₀ to test the inhibitory effect of the following synthesized tripeptides LKA-NH₂, ILK-NH₂, GPQ-NH₂, GHK-NH₂, GKG-NH₂, ACQ-NH₂, CQG-NH₂, ARV-NH₂, KAR-NH₂, HKA-NH₂, GAT-NH₂, KAL-NH₂, and GPG-NH₂. Accordingly, the H9 cells were resuspended with or without the different peptides (approximately 100 μM) in 1 ml of RPMI 1640 medium supplemented with 10% (v/v) heat-inactivated fetal bovine serum (FBS), penicillin (100 u/ml), and streptomycin (100 u/ml), all available through GIBCO, and Polybrene (2 μg/ml), available through Sigma. Thereafter, viruses were added at 25 TCID₅₀ in a volume of 20-30%1. Cells were incubated with virus at 37° C. for 1 hr then pelleted at 170× g for 7 minutes. The cells were then washed three times in RPMI medium without peptides at room temperature and pelleted at 170× g for 7 minutes, as above. After the final wash, the cells were resuspended in RPMI culture medium in a 24-well plate (Costar corporation) and kept at 37° C. in 5% CO₂ with humidity.

Culture supernatants were collected and analyzed when the medium was changed at 4, 7, 10, and 14 days post infection. To monitor the replication of virus, reverse transcriptase (RT) activity in the supernatants was assayed using a commercially available Lenti-RT activity kit. (Cavidi Tech, Uppsala, Sweden). The amount of RT was determined with the aid of a regression line of standards. The results are presented as absorbance values (OD) and higher absorbance indicates a higher protein concentration and greater viral infection. Syncytium formation was also monitored by microscopic examination. Tables 3 and 4 show the absorbance values of the cell culture supernatants of Experiments 1 and 2 respectively.

In Experiment 3, (Table 5), approximately 200,000H9 cells were infected with HIV-1, HIV-2 or SIV at 25 TCID₅₀ to test the inhibitory effect of different concentrations of peptides GPG-NH₂, GKG-NH₂ and CQG-NH₂ and combinations of these peptides (the indicated concentration corresponds to the concentration of each tripeptide). As above, H9 cells were resuspended with or without the different peptides at varying concentrations in 1 ml of RPMI 1640 medium supplemented with 10% (v/v) heat-inactivated fetal bovine serum (FBS), penicillin (100 u/ml), and streptomycin (lOOu/ml), and Polybrene (2 μg/ml). Thereafter, viruses were added at 25 TCID₅₀ in a volume of 20-30 μ. Cells were incubated with the indicated virus at 37° C. for 1 hr then pelleted at 170× g for 7 minutes. The cells were then washed three times in RPMI medium without peptides at room temperature and pelleted at 170× g for 7 minutes, as above. After the final wash, the cells were resuspended in RPMI culture medium in a 24-well plate (Costar corporation) and kept at 37° C. in 5% CO₂ with humidity.

Culture supernatants were collected when the medium was changed at 4, 7, and 11 days post infection. As above, the replication of each virus was monitored by detecting reverse transcriptase (RT) activity in the supernatants using the Lenti-RT activity kit. (Cavidi Tech). The amount of RT was determined with the aid of a regression line of standards. The results are presented as absorbance values (OD) and higher absorbance indicates a higher protein concentration and greater viral infection. Table 4 shows the absorbance values of the cell culture supernatents of Experiment 3.

In Experiment 4, (Table 6) approximately 200,000H9 cells were infected with HIV-1 at 25 TCID₅₀ to test the inhibitory effect of different concentrations of peptides GPG-NH₂, GKG-NH₂ and CQG-NH₂ and combinations of these peptides (the indicated concentration corresponds to the total concentration of tripeptide). As above, H9 cells were resuspended with or without the different peptides at varying concentrations in 1 ml of RPMI 1640 medium supplemented with 10% (v/v) heat-inactivated fetal bovine serum (FBS), penicillin (100 u/ml), and streptomycin (100 u/ml), and Polybrene (2 μg/ml). Thereafter, viruses were added at 25 TCID₅₀ in a volume of 20-30 μl. Cells were incubated with the indicated virus at 37° C. for 1 hr then pelleted at 170× g for 7 minutes. The cells were then washed three times in RPMI medium without peptides at room temperature and pelleted at 170× g for 7 minutes, as above. After the final wash, the cells were resuspended in RPMI culture medium in a 24-well plate (Costar corporation) and kept at 37° C. in 5% CO₂ with humidity.

Culture supernatants were collected when the medium was changed at 4, 7, and 11 days post infection. As above, the replication of each virus was monitored by detecting reverse transcriptase (RT) activity in the supernatants using the Lenti-RT activity kit. (Cavidi Tech). The amount of RT was determined with the aid of a regression line of standards. The results are presented as absorbance values (OD) and higher absorbance indicates a higher protein concentration and greater viral infection. Table 5 shows the absorbance values of the cell culture supernatents of Experiment 4. The supernatant analyzed at day 11 was diluted 5-fold so that detection could be more accurately determined.

In Experiment 5, (Table 7) approximately 200,000H9 cells were infected with HIV-1 at 25 TCID₅₀ to test the inhibitory effect of different concentrations of peptides GPG-NH₂, GKG-NH₂ and CQG-NH₂ and combinations of these peptides. As above, H9 cells were resuspended with or without the different peptides at varying concentrations in 1 ml of RPMI 1640 medium supplemented with 10% (v/v) heat-inactivated fetal bovine serum (FBS), penicillin (100 u/ml), streptomycin (100 u/ml), and Polybrene (2 μg/ml). Thereafter, viruses were added at 25 TCID₅₀ in a volume of 20-30 μl. Cells were incubated with the indicated virus at 37° C. for 1 hr then pelleted at 170× g for 7 minutes. The cells were then washed three times in RPMI medium without peptides at room temperature and pelleted at 170× g for 7 minutes, as above. After the final wash, the cells were resuspended in RPMI culture medium in a 24-well plate (Costar corporation) and kept at 37° C. in 5% CO₂ with humidity.

Culture supernatants were collected when the medium was changed at 4, 7, and 14 days post infection. The replication of each virus was monitored by detecting the presence of p24 in the supernatants. HIV p24 antigen was determined using a commercially available HIV p24 antigen detection kit (Abbott). The results are presented as absorbance values (OD) and higher absorbance indicates a higher protein concentration and greater viral infection. In some cases, serial dilutions of the supernatants were made so as to more accurately detect p24 concentration. Table 6 shows the absorbance values of the cell culture supernatants of Experiment 5. As discussed in greater detail below, it was discovered that the tripeptides GPG-NH₂, GKG-NH₂ and CQG-NH₂ and combinations of these peptides effectively inhibit HIV-1, HIV-2, and SIV infection.

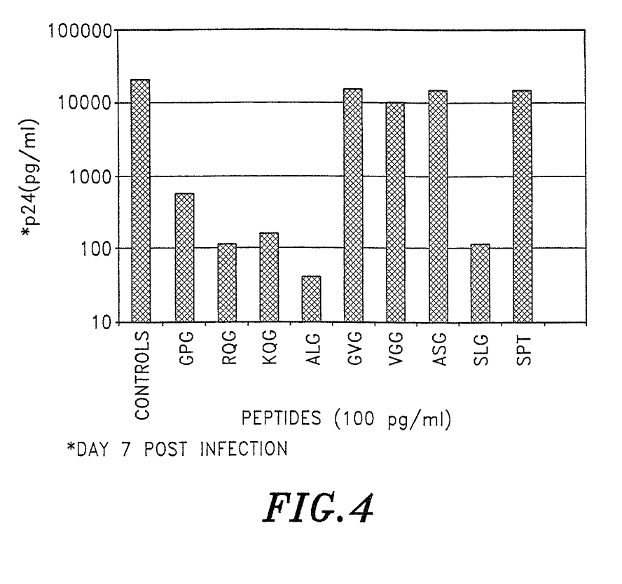

In experiment 6 (Table 8 and FIG. 4), approximately 200,000 HUT78 cells were infected with HIV-1 at 25 TCID₅₀ to test the inhibitory effect of GPG-NH₂, RQG-NH₂, KQG-NH₂, ALG-NH₂, GVG-NH₂, VGG-NH₂, ASG-NH₂, SLG-NH₂, and SPT-NH₂. The HUT cells were resuspended in 1 ml of RPMI 1640 medium supplemented with 10% (v/v) heat-inactivated fetal bovine serum (FBS, GIBCO), penicillin (100 u/ml), streptomycin (100 u/ml) and Polybrene (Sigma, 2 μg/ml) with or without the presence of the different small peptides (100 μM) mentioned above. Thereafter, the HIV-1 virus was added at 25 TCID₅₀ in a volume of 20 μl. Cells were incubated with the virus at 37° C. for one hour and, subsequently, the cells were pelleted at 170× g for seven minutes. The cells were then washed three times in RPMI medium without peptides at room temperature by cell sedimentation at 170× g for seven minutes, as above. After the final wash, the cells were resuspended in RPMI culture medium in 24-well plate (Costar corporation) and were kept at 37° C. in 5% CO₂ with humidity. Culture supernatants were collected when medium was changed at day 4, 7, and 11 post infection and viral p24 production was monitored by using an HIV-1 p24 ELISA kit (Abbott Laboratories, North Chicago, USA). As discussed below, it was discovered that the small peptides RQG-NH₂, KQG-NH₂, ALG-NH₂, GVG-NH₂, VGG-NH₂, ASG-NH₂, SLG-NH₂, and SPT-NH₂ effectively inhibit HIV-I infection.

TABLE 3
Experiment 1 - (peptides made on site)

| Tripeptide | Day 7 RT | | | Day 10 RT | | | HIV-1 |
|---|---|---|---|---|---|---|---|
| (100 μM) | HIV-1 | HIV-2 | SIV | HIV-1 | HIV-2 | SIV | Syncytia |
| LKA-NH₂ | 0.568* | 3.649 | 3.577 | 2.429 | 2.769 | 2.452 | pos |
| ILK-NH₂ | 0.365 | 3.467 | 3.180 | 2.033 | 2.791 | 2.255 | pos |
| GPQ-NH₂ | 0.204 | 3.692 | 1.542 | 1.965 | 2.734 | 2.176 | pos |
| GHK-NH₂ | 0.289 | 3.522 | 0.097 | 2.151 | 2.931 | 2.384 | pos |
| GKG-NH₂ | 0.080 | 0.160 | 0.421 | 0.074 | 0.147 | 0.099 | neg |
| ACQ-NH₂ | 0.117 | 3.418 | 1.241 | 0.904 | 2.753 | 2.746 | pos |
| CQG-NH₂ | 0.091 | 0.217 | 0.747 | 0.108 | 0.296 | 0.110 | neg |
| ARV-NH₂ | 0.156 | 3.380 | 0.210 | 1.528 | 3.003 | 1.172 | pos |
| KAR-NH₂ | 0.380 | 3.419 | 0.266 | 2.779 | 2.640 | 1.722 | pos |
| HKA-NH₂ | 0.312 | 3.408 | 0.416 | 2.546 | 2.669 | 2.520 | pos |
| GAT-NH₂ | 0.116 | 3.461 | 0.892 | 1.565 | 2.835 | 2.343 | pos |
| KAL-NH₂ | 0.246 | 3.372 | 1.091 | 1.995 | 2.749 | 2.524 | pos |
| GPG-NH₂ | 0.068 | 0.735 | 0.138 | 0.074 | 0.145 | 0.103 | neg |
| NO PEPTIDE | 0.251 | 1.675 | 1.227 | 2.217 | 2.657 | 3.030 | pos |
| CONTROL | | | | | | | |

* Values represent opitcal density (OD)

TABLE 4
Experiment 2 - (peptides made on site)

| Tripeptide | Day 7 RT | | | Day 10 RT | | | HIV-1 |
|---|---|---|---|---|---|---|---|
| (100 μM) | HIV-1 | HIV-2 | SIV | HIV-1 | HIV-2 | SIV | Syncytia |
| LKA-NH₂ | 0.894* | 1.689 | 0.724 | 2.989 | 2.637 | 2.797 | pos |
| ILK-NH₂ | 0.581 | 1.692 | 0.515 | 2.950 | 2.557 | 2.632 | pos |
| GPQ-NH₂ | 0.884 | 1.511 | 0.574 | 2.848 | 2.382 | 2.319 | pos |
| GHK-NH₂ | 0.829 | 1.936 | 0.396 | 3.013 | 2.418 | 2.394 | pos |
| GKG-NH₂ | 0.145 | 0.283 | 0.116 | 0.345 | 1.637 | 0.204 | neg |
| ACQ-NH₂ | 0.606 | 1.661 | 0.612 | 2.831 | 2.505 | 2.606 | pos |
| CQG-NH₂ | 0.143 | 1.241 | 0.120 | 1.546 | 2.501 | 1.761 | neg |
| ARV-NH₂ | 0.618 | 2.237 | 0.212 | 2.829 | 2.628 | 3.004 | pos |
| KAR-NH₂ | 0.753 | 1.904 | 1.034 | 2.928 | 2.742 | 2.672 | pos |
| HKA-NH₂ | 1.081 | 1.678 | 0.455 | 2.794 | 2.560 | 2.623 | pos |
| GAT-NH₂ | 0.776 | 1.707 | 0.572 | 2.800 | 2.565 | 2.776 | pos |
| KAL-NH₂ | 0.999 | 1.757 | 0.511 | 2.791 | 2.383 | 2.663 | pos |
| GPG-NH₂ | 0.090 | 0.093 | 0.067 | 0.143 | 0.575 | 0.139 | neg |
| NO PEPTIDE | 0.809 | 1.774 | 0.578 | 2.711 | 2.528 | 2.911 | pos |
| CONTROL | | | | | | | |

* Values represent opitcal density (OD)

TABLE 5
Experiment 3 - (peptides obtained from Bachem)

| | Day 7 RT | | | Day 10 RT | | |
|---|---|---|---|---|---|---|
| Tripeptide | HIV-1 | HIV-2 | SIV | HIV-1 | HIV-2 | SIV |
| NO PEPTIDE | 1.558* | 1.718 | 1.527 | 2.521 | 2.716 | 2.091 |
| CONTROL | | | | | | |
| GPG-NH₂ | 1.527 | 1.735 | 0.753 | 2.398 | 2.329 | 2.201 |
| 5 μM | | | | | | |
| GPG-NH₂ | 0.239 | 0.252 | 0.197 | 0.692 | 1.305 | 0.779 |
| 20 μM | | | | | | |
| GKG-NH₂ | 1.587 | 1.769 | 0.271 | 1.683 | 2.510 | 1.709 |
| 5 μM | | | | | | |
| GKG-NH₂ | 1.616 | 1.759 | 1.531 | 2.036 | 2.646 | 2.482 |
| 20 μM | | | | | | |
| GKG-NH₂ | 0.823 | 0.828 | 1.005 | 1.520 | 1.947 | 1.382 |
| 100 μM | | | | | | |
| CQG-NH₂ | 1.547 | 1.760 | 1.159 | 2.028 | 2.466 | 2.821 |
| 5 μM | | | | | | |
| CQG-NH₂ | 1.578 | 1.748 | 0.615 | 1.484 | 2.721 | 2.158 |
| 20 μM | | | | | | |
| CQG-NH₂ | 1.520 | 1.715 | 0.795 | 2.014 | 2.815 | 2.286 |
| 100 μM | | | | | | |
| GPG-NH₂ + | 1.430 | 1.738 | 1.131 | 1.998 | 2.770 | 2.131 |
| GKG-NH₂ | | | | | | |
| 5 μM | | | | | | |
| GPG-NH₂ + | 0.129 | 0.244 | 0.123 | 0.164 | 1.110 | 0.309 |
| GKG-NH₂ | | | | | | |
| 20 μM | | | | | | |
| GPG-NH₂ + | 1.605 | 1.749 | 1.737 | 1.866 | 2.814 | 2.206 |
| CQG-NH₂ | | | | | | |
| 5 μM | | | | | | |
| GPG-NH₂ + | 0.212 | 0.194 | 0.523 | 0.397 | 1.172 | 0.910 |
| CQG-NH₂ | | | | | | |
| 20 μM | | | | | | |
| GKG-NH₂ + | 1.684 | 1.717 | 1.725 | 1.848 | 2.778 | 2.949 |
| CQG-NH₂ | | | | | | |
| 5 μM | | | | | | |
| GKG-NH₂ + | 1.490 | 1.792 | 1.670 | 1.891 | 2.799 | 2.889 |
| CQG-NH₂ | | | | | | |
| 20 μM | | | | | | |
| GPG-NH₂ + | 1.652 | 1.743 | 1.628 | 1.999 | 2.777 | 2.659 |
| GKG-NH₂ | | | | | | |
| 5 μM | | | | | | |
| GPG-NH₂ + | 0.165 | 0.119 | 0.317 | 0.307 | 0.447 | 0.389 |
| GKG-NH₂ | | | | | | |
| 20 μM | | | | | | |

* Values represent opitcal density (OD)

TABLE 6
Experiment 4 - (peptides obtained from Bachem)

| | | Day 10 RT |
|---|---|---|
| | Day 7 RT | HIV-1 |
| Tripeptide | HIV-1 | (1:5) |
| NO PEPTIDE CONTROL | 3.288* | 1.681 |
| GPG 5 μM | 2.970 | 1.107 |
| GPG 15 μM | 0.894 | 0.095 |
| GPG 45 μM | 0.177 | 0.034 |
| GPG 100 μM | 0.150 | 0.033 |
| GKG 5 μM | 3.303 | 1.287 |
| GKG 15 μM | 3.551 | 1.530 |
| GKG 45 μM | 3.126 | 0.410 |
| CQG 5 μM | 2.991 | 1.459 |
| CQG 15 μM | 2.726 | 1.413 |
| CQG 45 μM | 3.124 | 1.364 |
| GPG-NH₂ + GKG-NH₂ | 2.266 | 0.438 |
| 5 μM | | |
| GPG-NH₂ + GKG-NH₂ | 0.216 | 0.044 |
| 15 μM | | |
| GPG-NH₂ + CQG-NH₂ | 2.793 | 0.752 |
| 5 μM | | |
| GPG-NH₂ + CQG-NH₂ | 0.934 | 0.110 |
| 15 μM | | |
| GkG-NH₂ + CQG-NH₂ | 3.534 | 1.305 |
| 5 μM | | |
| GKG-NH₂ + CQG-NH₂ | 3.355 | 2.013 |
| 15 μM | | |
| GPG-NH₂ + GKG-NH₂ + CQG-NH₂ | 2.005 | 0.545 |
| 5 μM | | |
| GPG-NH₂ + GKG-NH₂ + CQG-NH₂ | 0.851 | 0.110 |
| 15 μM | | |

* Values represent optical density (OD)

TABLE 7
Experiment 5 - (peptides made on site)

| Tripeptide (μM) | p24 (OD) | p24 (pg/ml) | reduction (%) |
|---|---|---|---|
| Day 7 HIV-1 | | | |
| NO PEPTIDE CONTROL | 1.093 × 10² | 3.94 × 10⁴ | 0 |
| GPG-NH₂ (20) | 1.159 | 4.21 × 10² | 99 |
| GPG-NH₂ (100) | 0.508 | 1.60 × 10² | 100 |
| GPG-NH₂ (300) | 0.557 | 1.80 × 10² | 100 |
| GKG-NH₂ (100) | 0.566 × 10¹ | 1.83 × 10³ | 95 |
| GKG-NH₂ (300) | 1.08 | 3.88 × 10² | 99 |
| GKG-NH₂ (1000) | 0.79 | 2.73 × 10² | 100 |
| CQG-NH₂ (100) | 1.51 × 10¹ | 5.62 × 10³ | 86 |
| CQG-NH₂ (300) | 0.59 × 10¹ | 1.92 × 10³ | 95 |
| CQG-NH₂ (1000) | 0.91 | 3.20 × 10² | 99 |
| combined* | 0.65 | 2.17 × 10² | 100 |
| Day 14 HIV-1 | | | |
| NO PEPTIDE CONTROL | 0.46 × 10⁴ | 1.41 × 10⁶ | 0 |
| GPG-NH₂ (20) | 1.12 × 10² | 4.06 × 10⁴ | 97 |
| GPG-NH₂ (100) | 1.76 | 6.63 × 10² | 100 |
| GPG-NH₂ (300) | 1.35 | 4.98 × 10² | 100 |
| GKG-NH₂ (100) | 1.48 × 10³ | 5.51 × 10⁵ | 61 |
| GKG-NH₂ (300) | 0.33 × 10¹ | 8.70 × 10² | 100 |
| GKG-NH₂ (1000) | 0.11 × 10¹ | 2.40 × 10² | 100 |
| CQG-NH₂ (100) | 0.48 × 10⁴ | 1.47 × 10⁶ | 0 |
| CQG-NH₂ (300) | 0.11 × 10² | 2.40 × 10³ | 100 |
| CQG-NH₂ (1000) | 0.13 × 10¹ | 2.80 × 10² | 100 |
| combined* | 1.01 | 3.61 × 10² | 100 |

*100 μM GPG-NH₂ + GKG-NH₂ + CQG-NH₂
* Values represent opitcal density (OD)

TABLE 8
Experiment 6 - (peptides made on site)

| Tripeptide (100 μM) | p24 (pg/ml) | reduction (%) |
|---|---|---|
| Day 7 HIV-1 | | |
| NO PEPTIDE CONTROL | 2.0 × 10⁴ | 0 |
| GPG-NH₂ | 5.6 × 10² | 97 |
| RQG-NH₂ | 1.13 × 10² | 99 |
| KQG-NH₂ | 1.54 × 10² | 99 |
| ALG-NH₂ | 0.42 × 10² | 100 |
| GVG-NH₂ | 1.5 × 10⁴ | 25 |
| VGG-NH₂ | 1.0 × 10⁴ | 50 |
| ASG-NH₂ | 1.5 × 10⁴ | 25 |
| SLG-NH₂ | 1.14 × 10² | 99 |
| SPT-NH₂ | 1.5 × 10⁴ | 25 |

Of the small peptides listed in Table 1, GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂ inhibited and/or prevented HIV-1 infection and GKG-NH₂, CQG-NH₂, and GPG-NH2 were also shown to inhibit or prevent HIV-2 and SIV infection. It should be understood that the small peptides RQG-NH₂, KQG-NH₂, ALG-NH₂, GVG-NH₂, VGG-NH₂, ASG-NH₂, SLG-NH₂, and SPT-NH₂ were not analyzed for their ability to prevent or inhibit HIV-2 or SIV infection but, given the fact that HIV-2 and SIV share significant homology in capsid protein structure at the region to which the small peptides GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂ correspond, an inhibition or prevention of HIV-2 or SIV infection or both is expected.

The results for Experiments 1-6 (shown in Tables 3-8 and FIG. 4), demonstrate that small peptides in amide form that correspond to viral capsid protein sequence having a glycine as the carboxyterminal amino acid, GPG-NH₂, GKG-NH₂, CQG-NH₂, RQG-NH₂, KQG-NH₂, ALG-NH₂, GVG-NH₂, VGG-NH₂, ASG-NH₂, and SLG-NH₂, inhibited or prevented HIV infection. Peptides containing a carboxyterminal alanine residue, Leu-Lys-Ala (LKA) and His-Lys-Ala (HKA) or a carboxyterminal glutamine residue, Gly-Pro-Gln (GPQ) and Ala-Cys-Gln (ACQ) did not prevent HIV infection. Glycine at the amino terminus was not an inhibitory factor, however, because the peptides with an amino terminal glycine residue, Gly-Pro-Gln (GPQ), Gly-His-Lys (GHK), and Gly-Ala-Thr (GAT) failed to prevent infection and syncytia formation. Further, peptides with other uncharged polar side chains such as Gly-Pro-Gln (GPQ), Ala-Cys-Gln (ACQ), and Gly-Ala-Thr (GAT) or non-polar side chains at the carboxy terminus such as Ala-Arg-Val (ARV), His-Lys-Ala (HKA), and Lys-Ala-Leu (KAL), and Leu-Lys-Ala (LKA) failed to prevent infection. Although a glycine residue at the carboxy terminus appears to be associated with the inhibition of HIV and SIV infection, other amino acid residues or modified amino acid residues at the carboxy terminus of a small peptide can also inhibit HIV and SIV infection. For example, it was shown that Ser-Pro-Thr (SPT) inhibited or prevented HIV-1 infection.

In some experiments, the effect of the small peptides on HIV-1, HIV-2, and SIV infection was concentration and time dependent. Concentrations of GKG-NH₂, CQG-NH₂, and GPG-NH₂ and combinations thereof, as low as 5 μM and 20 μM were effective at reducing HIV-1, HIV-2, and SIV infection. At 100 μM or greater, however, the tripeptides GKG-NH₂, CQG-NH₂, and GPG-NH₂ and combinations thereof more efficiently inhibited HIV-, HIV-2, and SIV infection. As shown in Table 7, 300 μM of GKG-NH2 and CQG-NH2 reduced HIV-1 infectivity by almost 100%, as detected by the presence of p24 antigen in cell supematents. The percent reduction tabulated in Table 7 was calculated by dividing amount of p24 antigen detected in the peptide-treated sample by the amount of p24 antigen detected in the control sample, multiplying this dividend by 100 to obtain a percentage, and subtracting the dividend percentage by 100%. For example, the percent reduction exhibited by GPG-NH₂ is:

$$\frac{5.6 \times {10}^{2}}{2.0 \times {10}^{4}} \times 100 = 3 \% and 100 \% - 3 \% = 97 \% .$$

In the first five experiments (Tables 3-7) it was shown that the tripeptides GKG-NH₂, CQG-NH₂, and GPG-NH₂ and combinations thereof, inhibit HIV-1, HIV-2, and SIV infection at concentrations equal to or greater than 5 μM.

In the sixth experiment (Table 8 and FIG. 4), it was shown that the small peptides RQG-NH₂, KQG-NH₂, ALG-NH₂, GVG-NH₂, VGG-NH₂, ASG-NH₂, SLG-NH₂, and SPT-NH₂ effectively inhibit and/or prevent HIV-1 infection at 100 μM. As shown in Table 7, a nearly 100% reduction of virus, as measured by the amount of capsid protein p24 in the supernatent, was achieved with the small peptides RQG-NH₂, KQG-NH₂, ALG-NH₂, and SLG-NH₂. The percent reduction of p24 shown in Table 8 was calculated as described for Table 7, above. Although GVG-NH₂, VGG-NH₂, ASG-NH₂, and SPT-NH₂ were less effective at inhibiting or preventing HIV-1 infection at 100 μM, it is believed that the tripeptides are more effective at higher concentrations. The data presented in experiments 1-6, shown in Tables 3-8 and FIG. 4, demonstrate that small peptides that correspond to sequences of a viral capsid protein are effective antiviral agents over a wide-range of concentrations.

In the experiments above, it has been demonstrated that modified small peptides having a sequence that corresponds to viral capsid proteins inhibit viral infection (e.g., HIV-1, HIV-2, and SIV infection) by binding to the viral capsid protein, preventing or inhibiting viral capsid protein polymerization and, thereby, interrupting proper capsid assembly and viral infection. The many assays detailed above can be used to identify the ability of any small peptide, modified small peptide, oligopeptide, or peptidomimetic to prevent or inhibit HIV or SIV infection. Similar techniques can also be used to identify the ability of any small peptide, modified small peptide, oligopeptide, or peptidomimetic to prevent or inhibit other viral infections. Further, this group of experiments provides another example of peptide agents that are effective inhibitors of the protein-protein interactions that are necessary for protein polymerization.

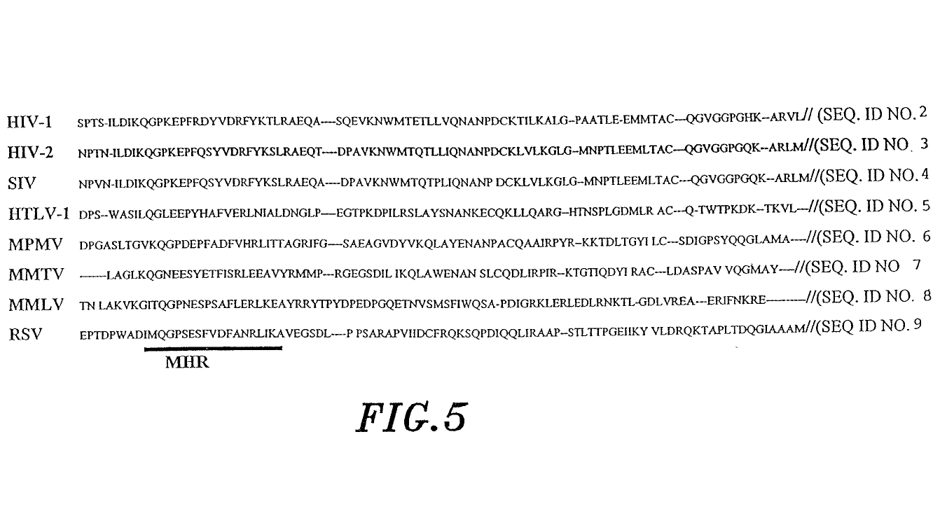

Because the sequence of several viral capsid proteins are known, the design, manufacture, and identification of small peptides in amide form that prevent proper polymerization of different viral capsid proteins is straightforward. Several viral capsid proteins, for instance, contain a 20 amino acid long homology region called the major homology region (MHR), that exists within the carboxyl-terminal domain of many onco- and lentiviruses. (See FIG. 5). FIG. 5 shows the carboxyl-terminal domain of HIV-1 (residues 146-231) and compares this sequence to the capsid protein sequences of other viruses, some of which infect birds, mice, and monkeys. Notably, considerable homology in the sequences of these viral capsid proteins is found. Investigators have observed that the carboxyl-terminal domain is required for capsid dimerization and viral assembly in HIV-1. (Gamble et al., Science 278: 849 (1997), herein incorporated by reference). While the small peptides that exhibited antiviral activity in the assays described in this disclosure fully or partially corresponded to regions of the carboxyl-terminal domain of HIV-1, HIV-2, or SIV, regions of the N-terminal domain of viruses are important for capsid polymerization and the design and synthesis of small peptides that either fully or partially correspond to amino acids of the N-terminal region of viral capsid proteins are desirable embodiments of the present invention. The use of small peptides that fully or partially correspond to amino acids within the MHR region and the carboxyl-terminal domain of viral capsid proteins, however, are preferred embodiments of the present invention.

By designing and manufacturing small peptides, oligopeptides, and/or peptidomimetics that correspond to regions of the sequences disclosed in FIG. 5, new molecules that inhibit HIV, SIV, RSV, HTLV-1, MMTV, MPMV, and MMLV infection can be rapidly identified by using the screening techniques discussed above or modifications of these assays, as would be apparent to one of skill in the art. Further, many of the sequences of other viral capsid proteins are known, such as members of the arenavirus, rotavirus, orbivirus, retrovirus, papillomavirus, adenovirus, herpesvirus, paramyxovirus, myxovirus, and hepadnavirus families. Several small peptides, oligopeptides, and/or peptidomimetics that fully or partially correspond to these sequences can be selected and rapidly screened to identify those that effectively inhibit and/or prevent viral infection by using the viral infectivity assays, viral capsid protein binding assay, and electron microscopy techniques described herein, or modifications of these assays as would be apparent to those of skill in the art given the present disclosure.

Desirable embodiments are peptide agents, which include small peptides (more than one amino acid and less than or equal to 10 amino acids in length) having a modified carboxy terminus that are used to interrupt protein-protein interactions, protein polymerization, and the assembly of supramolecular complexes. Preferably, dipeptides, tripeptides, and oligopetides and corresponding peptidomimetics having a sequence that corresponds to a region of a protein involved in a protein-protein interaction, protein polymerization event, or assembly of a supramolecular complex are used. For example, an oligopeptide of the present invention may have four amino acids, five amino acids, six amino acids, seven amino acids, eight, or nine or ten amino acids and peptidomimetics of the present invention may have structures that resemble four, five, six, seven, eight, nine, or ten amino acids. Desirable oligopeptides can include the full or partial sequences found in the tripeptides GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂. Peptidomimetics that resemble dipeptides, tripeptides and oligopeptides also, can correspond to a sequence that is found in GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂.

It is preferred that the small peptides possess a modulation group (e.g., an amide group) at their carboxy termini (CO-NH₂) rather than a carboxyl group (COOH). Small peptides having other modulation groups at the carboxy terminus, can also be used but desirably, the attached modulation groups have the same charge and sterically behave the same as an amide group. (See U.S. Pat. No. 5,627,035 to Vahlne et al., for an assay to compare peptides having differing substituents at the carboxyl terminus). Unexpectedly, the inventor has discovered that a modulation group (e.g., an amide group or a substituent that chemically and sterically behaves like an amide group), allows the peptide agent to interact with the protein of interest and, thereby, interrupt protein-protein interactions, protein polymerization, and the assembly of supramolecular complexes.

In the following disclosure, several approaches are provided to make biotechnological tools and pharmaceutical compositions comprising dipeptides, tripeptides, oligopeptides of less than or equal to 10 amino acids, and peptidomimetics that resemble tripeptides and oligopeptides of less than or equal to 10 amino acids (collectively referred to as a “peptide agent(s)”). It should be noted that the term “peptide agents” includes dipeptides, tripeptides, and oligopeptides of less than or equal to 10 amino acids. “Peptide agents” are, for example, peptides of two, three, four, five, six, seven, eight, nine, or ten amino acids and peptidomimetics that resemble peptides of two, three, four, five, six, seven, eight, nine, or ten amino acids. Further, “peptide agents” are peptides of two, three, four, five, six, seven, eight, nine, or ten amino acids or peptidomimetics that resemble two, three, four, five, six, seven, eight, nine, or ten amino acids that are provided as multimeric or multimerized agents, as described below.

Desirable biotechnological tools or components to prophylactic or therapeutic agents, provide the peptide agent in such a form or in such a way that a sufficient affinity or inhibition of a protein-protein interaction, protein polymerization event, or assembly of supramolecular complex is obtained. While a natural monomeric peptide agent (e.g., appearing as discrete units of the peptide agent each carrying only one binding epitope) can be sufficient, synthetic ligands or multimeric ligands (e.g., appearing as multiple units of the peptide agent with several binding epitopes) can have far greater capacity to inhibit protein-protein interactions, protein polymerization, and the assembly of supramolecular complexes. It should be noted that the term “multimeric” is meant to refer to the presence of more than one unit of a ligand, for example several individual molecules of a tripeptide, oligopeptide, or a peptidomimetic, as distinguished from the term “multimerized” that refers to the presence of more than one ligand joined as a single discrete unit, for example several tripeptides, oligopeptides, or peptidomimetic molecules joined in tandem.

A multimeric agent (synthetic or natural) can be obtained by coupling a peptide agent to a macromolecular support. A “support” can also be termed a carrier, a resin or any macromolecular structure used to attach, immobilize, or stabilize a peptide agent. Solid supports include, but are not limited to, the walls of wells of a reaction tray, test tubes, polystyrene beads, magnetic beads, nitrocellulose strips, membranes, microparticles such as latex particles, sheep (or other animal) red blood cells, artificial cells and others. Supports are also carriers as understood for the preparation of pharmaceuticals.

The macromolecular support can have a hydrophobic surface that interacts with a portion of the peptide agent by hydrophobic non-covalent interaction. The hydrophobic surface of the support can also be a polymer such as plastic or any other polymer in which hydrophobic groups have been linked such as polystyrene, polyethylene or polyvinyl. Alternatively, the peptide agent can be covalently bound to carriers including proteins and oligo/polysaccarides (e.g. cellulose, starch, glycogen, chitosane or aminated sepharose). In these later embodiments, a reactive group on the peptide agent, such as a hydroxy or an amino group, can be used to join to a reactive group on the carrier so as to create the covalent bond. The support can also have a charged surface that interacts with the peptide agent. Additionally, the support can have other reactive groups that can be chemically activated so as to attach a peptide agent. For example, cyanogen bromide activated matrices, epoxy activated matrices, thio and thiopropyl gels, nitrophenyl chloroformate and N-hydroxy succinimide chlorformate linkages, and oxirane acrylic supports are common in the art.

The support can also comprise an inorganic carrier such as silicon oxide material (e.g. silica gel, zeolite, diatomaceous earth or aminated glass) to which the peptide agent is covalently linked through a hydroxy, carboxy or amino group and a reactive group on the carrier. Furthermore, in some embodiments, a liposome or lipid bilayer (natural or synthetic) is contemplated as a support and peptide agents are attached to the membrane surface or are incorporated into the membrane by techniques in liposome engineering. By one approach, liposome multimeric supports comprise a peptide agent that is exposed on the surface of the bilayer and a second domain that anchors the peptide agent to the lipid bilayer. The anchor can be constructed of hydrophobic amino acid residues, resembling known transmembrane domains, or can comprise ceramides that are attached to the first domain by conventional techniques.

Supports or carriers for use in the body, (i.e. for prophylactic or therapeutic applications) are desirably physiological, non-toxic and preferably, non-immunoresponsive. Contemplated carriers for use in the body include poly-L-lysine, poly-D, L-alanine, liposomes, and Chromosorb® (Johns-Manville Products, Denver Co.). Ligand conjugated Chromosorb® (Synsorb-Pk) has been tested in humans for the prevention of hemolytic-uremic syndrome and was reported as not presenting adverse reactions. (Armstrong et al. J. Infectious Diseases, 171:1042-1045 (1995)). For some embodiments, the present inventor contemplates the administration of a “naked” carrier (i.e., lacking an attached peptide agent) that has the capacity to attach a peptide agent in the body of a subject. By this approach, a “prodrug-type” therapy is envisioned in which the naked carrier is administered separately from the peptide agent and, once both are in the body of the subject, the carrier and the peptide agent are assembled into a multimeric complex.

The insertion of linkers, such as λ linkers, of an appropriate length between the peptide agent and the support are also contemplated so as to encourage greater flexibility of the peptide agent and thereby overcome any steric hindrance that may be presented by the support. The determination of an appropriate length of linker can be determined by screening the peptide agents with varying linkers in the assays detailed in the present disclosure.

A composite support comprising more than one type of peptide agent is also an embodiment. A “composite support” can be a carrier, a resin, or any macromolecular structure used to attach or immobilize two or more different peptide agents that bind to a capsomere protein, such as p24, and/or interfere with capsid assembly and/or inhibit viral infection, such as HIV or SIV infection. In some embodiments, a liposome or lipid bilayer (natural or synthetic) is contemplated for use in constructing a composite support and peptide agents are attached to the membrane surface or are incorporated into the membrane using techniques in liposome engineering. As above, the insertion of linkers, such as λ linkers, of an appropriate length between the peptide agent and the support is also contemplated so as to encourage greater flexibility in the molecule and thereby overcome any steric hindrance that may occur. The determination of an appropriate length of linker can be determined by screening the ligands with varying linkers in the assays detailed in the present disclosure.

In other embodiments of the present invention, the multimeric and composite supports discussed above can have attached multimerized ligands so as to create a “multimerized-multimeric support” and a “multimerized-composite support”, respectively. A multimerized ligand can, for example, be obtained by coupling two or more peptide agents in tandem using conventional techniques in molecular biology. The multimerized form of the ligand can be advantageous for many applications because of the ability to obtain an agent with a better ability to bind to a capsomere protein, such as p24, and/or interfere with capsid assembly and/or inhibit viral infection, such as HIV or SIV infection. Further, the incorporation of linkers or spacers, such as flexible λ linkers, between the individual domains that make-up the multimerized agent is an advantageous embodiment. The insertion of A linkers of an appropriate length between protein binding domains, for example, can encourage greater flexibility in the molecule and can overcome steric hindrance. Similarly, the insertion of linkers between the multimerized ligand and the support can encourage greater flexibility and limit steric hindrance presented by the support. The determination of an appropriate length of linker can be determined by screening the ligands with varying linkers in the assays detailed in this disclosure.

In preferable embodiments, the various types of supports discussed above are created using the modified tripeptides GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂. The multimeric supports, composite supports, multimerized-multimeric supports, or multimerized-composite supports, collectively referred to as “support-bound agents”, are also preferably constructed using the tripeptides GPG-NH₂, GKG-NH₂, CQG-NH₂, RQG-NH₂, KQG-NH₂, ALG-NH₂, GVG-NH₂, VGG-NH₂, ASG-NH₂, SLG-NH₂, and SPT-NH₂.

Several methods of making and using the compositions disclosed herein are also embodiments. By one approach, peptide agents obtained by PPI technology are incorporated into pharmaceuticals. That is, peptide agents that are selected, designed, manufactured, and identified for their ability to prevent or inhibit protein-protein interactions, protein polymerization events, or disease (e.g., peptide agents identified by their performance in peptide characterization assays) are incorporated into pharmaceuticals for use in treating human disease. In some aspects, selection and design is accomplished with the aid of a computer system. Search programs and retrieval programs, for example, are used to access one or more databases to select and design peptide agents that inhibit protein-protein interactions, protein polymerization, or supramolecular complex assembly. Additionally, approaches in rational drug design, as described above, are used to select and design peptide agents. Once selected and designed, the peptide agent is “obtained” (e.g., manufactured or purchased from a commercial entity). Next, the peptide agent is screened in peptide characterization assays that assess the ability of the peptide agent to bind to a protein of interest, interrupt protein polymerization, and prevent or treat disease. Peptide agents are then selected on the basis of their performance in such characterization assays. Profiles having a symbol that represents the peptide agent and one or more symbols representing a performance on a peptide characterization assay can be created and these profiles can be compared to select an appropriate peptide agent for incorporation into a pharmaceutical or for further selection and design of new peptide agents. Once characterized, the peptide agents are incorporated into a pharmaceutical according to conventional techniques.

The pharmacologically active compounds can be processed in accordance with conventional methods of galenic pharmacy to produce medicinal agents for administration to patients, e.g., mammals including humans. The peptide agents can be incorporated into a pharmaceutical product with and without modification. Further, the manufacture of pharmaceuticals or therapeutic agents that deliver the peptide agent or a nucleic acid sequence encoding a small peptide by several routes is an embodiment. For example, and not by way of limitation, DNA, RNA, and viral vectors having sequence encoding a small peptide that interrupts a protein-protein interaction, a protein polymerization event, or the assembly of a supramolecular complex are within the scope of aspects of the present invention. Nucleic acids encoding a desired peptide agent can be administered alone or in combination with peptide agents.

The peptide agents can be employed in admixture with conventional excipients, i.e., pharmaceutically acceptable organic or inorganic carrier substances suitable for parenteral, enteral (e.g., oral) or topical application that do not deleteriously react with the peptide agents. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds. They can also be combined where desired with other active agents, e.g., vitamins.

The effective dose and method of administration of a particular peptide agent formulation may vary based on the individual patient and the stage of the disease, as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, age, weight and gender of the patient; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Normal dosage amounts may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, and 10 g. Additionally, the concentrations of the peptide agents of the present invention can be quite high in embodiments that administer the agents in a topical form. Molar concentrations of peptide agents can be used with some embodiments. Desirable concentrations for topical administration and/or for coating medical equipment range from 100 μM to 800 mM. Preferable concentrations for these embodiments range from 500 μM to 500 mM. For example, preferred concentrations for use in topical applications and/or for coating medical equipment include 500 μM, 550 μM, 600 μM, 650 μM, 700 μM, 750 μM, 800 μM, 850 μM, 900 μM, 1 mM, 5 mM, 10 mM, 15 mM, 20 mM, 25 mM, 30 mM, 35 mM, 40 mM, 45 mM, 50 mM, 60 mM, 70 mM, 80 mM, 90 mM, 100 mM, 120 mM, 130 mM, 140 mM, 150 mM, 160 mM, 170 mM, 180 mM, 190 mM, 200 mM, 300 mM, 325 mM, 350 mM, 375 mM, 400 mM, 425 mM, 450 mM, 475 mM, and 500 mM. Guidance as to particular dosages and methods of delivery is provided in the literature, (see e.g., U.S. Pat. Nos. 4,657,760; 5,206,344; or 5,225,212) and below.

More specifically, the dosage of the peptide agents of the present invention is one that provides sufficient peptide agent to attain a desirable effect. Accordingly, the dose of embodiments of the present invention may produce a tissue or blood concentration or both from approximately 0.1 μM to 500 mM. Desirable doses produce a tissue or blood concentration or both of about 1 to 800 μM. Preferable doses produce a tissue or blood concentration of greater than about 10 μM to about 500 μM. Preferable doses are, for example, the amount of small peptide required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration of greater than 800 μM are not preferred, they can be used with some embodiments of the present invention. A constant infusion of the peptide can also be provided so as to maintain a stable concentration in the tissues as measured by blood levels.

Routes of administration of the peptide agents include, but are not limited to, topical, transdermal, parenteral, gastrointestinal, transbronchial, and transalveolar. Topical administration is accomplished via a topically applied cream, gel, rinse, etc. containing a peptide. Transdermal administration is accomplished by application of a cream, rinse, gel, etc. capable of allowing the peptide agent to penetrate the skin and enter the blood stream. Parenteral routes of administration include, but are not limited to, electrical or direct injection such as direct injection into a central venous line, intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally.

Compositions of peptide agent-containing compounds suitable for topical application include, but not limited to, physiologically acceptable implants, ointments, creams, rinses, and gels. Any liquid, gel, or solid, pharmaceutically acceptable base in which the peptides are at least minimally soluble is suitable for topical use in the present invention. Compositions for topical application are particularly useful during sexual intercourse to prevent transmission of HIV. Suitable compositions for such use include, but are not limited to, vaginal or anal suppositories, creams, and douches.

Compositions of the peptide agents suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al., herein incorporated by reference.

Compositions of the peptide agents suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, or subcutaneous injection of the peptide agents.

Compositions of the peptide agents suitable for transbronchial and transalveolar administration include, but not limited to, various types of aerosols for inhalation. For instance, pentamidine is administered intranasally via aerosol to AIDS patients to prevent pneumonia caused by pneumocystis carinii. Devices suitable for transbronchial and transalveolar administration of the peptides are also embodiments. Such devices include, but are not limited to, atomizers and vaporizers. Many forms of currently available atomizers and vaporizers can be readily adapted to deliver peptide agents.

Compositions of the peptide agents suitable for gastrointestinal administration include, but not limited to, pharmaceutically acceptable powders, pills or liquids for ingestion and suppositories for rectal administration. Due to the most common routes of HIV infection and the ease of use, gastrointestinal administration, particularly oral, is the preferred embodiment of the present invention. Five-hundred milligram capsules having a tripeptide (GPG-NH₂) have been prepared and were found to be stable for a minimum of 12 months when stored at 4° C. As previously shown in other virus-host systems, specific antiviral activity of small peptides can be detected in serum after oral administration. (Miller et al., Appl. Microbiol., 16:1489 (1968)).

The peptide agents are also suitable for use in situations where prevention of HIV infection is important. For instances, medical personnel are constantly exposed to patients who may be HIV positive and whose secretions and body fluids contain the HIV virus. Further, the peptide agents can be formulated into antiviral compositions for use during sexual intercourse so as to prevent transmission of HIV. Such compositions are known in the art and also described in international application published under the PCT publication number WO90/04390 on May 3, 1990 to Modak et al., which is incorporated herein by reference.

Aspects of the invention also include a coating for medical equipment such as gloves, sheets, and work surfaces that protects against HIV transmission. Alternatively, the peptide agents can be impregnated into a polymeric medical device. Particularly preferred are coatings for medical gloves and condoms. Coatings suitable for use in medical devices can be provided by a powder containing the peptides or by polymeric coating into which the peptide agents are suspended. Suitable polymeric materials for coatings or devices are those that are physiologically acceptable and through which a therapeutically effective amount of the peptide agent can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, issued Sep. 16, 1986 to Fox et al. that is incorporated herein by reference.

The monomeric and multimeric peptide agents are suitable for treatment of subjects either as a preventive measure or as a therapeutic to treat subjects already afflicted with disease. Thus, methods of treatment of human disease are embodiments of the invention. Although anyone could be treated with the peptides as a prophylactic, the most suitable subjects are people at risk for contracting a particular disease. In many methods of the invention, for example, an individual at risk is first identified.

Individuals suffering from an NFκB-related disease (e.g., inflammatory disease or immune disorder) can be identified based on the expression levels of a gene product associated with this transcriptional activator. Individuals having an overexpression of a cytokine, for example, can be identified by a protein-based or RNA-based diagnostic. Once identified, the individual is administered a therapeutically effective dose of a peptide agent that inhibits dimerization of NFκB. In a similar fashion, individuals that overexpress IκB can be treated. Accordingly, individuals are identified by a protein-based or RNA-based diagnostic and once identified, the individual is administered a therapeutically effective amount of a peptide agent that disrupts formation of the NFκB/IκB complex.

Further, individuals suffering from the toxic effects of a bacterial toxin can be treated. Although peptide agents can be administered to anyone, as a preventative, for amelioration of the toxic effects of a bacterial toxin, preferably, infected individuals or persons at risk of bacterial infection are identified. Many diagnostic tests that can make this determination are known in the art. Once identified, the individual is administered a therapeutically effective amount of a peptide agent that interrupts the formation of a bacterial holotoxin.

Additional embodiments include methods of treatment and prevention of Alzheimers disease and scrapie. Although many people can be at risk for contracting these diseases and can be identified on this basis, individuals having a family history or a genetic marker associated with Alzheimer's disease or who have tested positive for the presence of the prion-related protein are preferably identified as patients at risk. Several diagnostic approaches to identify persons at risk of developing Alzheimer's disease have been reported. (See e.g., U.S. Pat. Nos., 5,744,368; 5,837,853; and 5,571,671). These approaches can be used to identify a patient at risk of developing Alzheimer's or others known to those of skill in the art can be employed. Once identified, an individual afflicted with Alzheimer's disease or a patient at risk of having Alzheimer's disease is administered a therapeutically safe and effective amount of a peptide agent that has been selected, designed, manufactured, and characterized by the approaches detailed above (collectively referred to as “PPI technology”). Similarly, when a person has been identified as having evidence of prion-related protein, PPI technology is used to generate a pharmaceutical that is administered to the subject in need so as to treat the condition.

An additional embodiment of the invention is a method of treatment or prevention of cancer in which a patient afflicted with cancer or a patient at risk of having cancer is identified and then is administered a therapeutically safe and effective amount of a peptide agent obtained by PPI technology. This method can be used to treat or prevent many forms of cancer associated with tubulin polymerization including but not limited to leukemia, prostate cancer, and colon cancer. Although, in some contexts, everyone is at risk of developing cancer and therefore are identified as individuals in need of treatment, desirably individuals with a medical history or family history are identified for treatment. Several diagnostic procedures for determining whether a person is at risk of developing different forms of cancer are available. For example, U.S. Pat. No. 5,891,857 provides approaches to diagnose breast, ovarian, colon, and lung cancer based on BRCA1 detection, U.S. Pat. No. 5,888,751 provides a general approach to detect cell transformation by detecting the SCP-1, marker, U.S. Pat. No. 5,891,651 provides approaches to detect colorectal neoplasia by recovering colorectal epithelial cells or fragments thereof from stool, U.S. Pat. No. 5,902,725 provides approaches to detect prostate cancer by assaying for the presence of a prostate specific antigen having a linked oligosaccharide that is triantennary, and U.S. Pat. No. 5,916,751 provides approaches to diagnose mucinous adenocarcinoma of the colon or ovaries, or an adenocarcinoma of the testis by detecting the presence of the TGFB-4 gene. Many more genetic based and blood based screens are known.

Further, methods of treatment of viral disease are provided. Accordingly, an infected individual is identified and then is administered a therapeutically effective amount of a peptide agent that interrupts viral capsid assembly and, thus, viral infection. Indivisuals having viral infection or those at risk of viral infection are preferably identified as subjects in need.

Additionally, in some embodiments, the peptide agents are administered in conjunction with other conventional therapies for the treatment of human disease. By one approach, peptide agents are administered in conjunction with a cytoreductive therapy (e.g., surgical resection of the tumor) so as to achieve a better tumorcidal response in the patient than would be presented by surgical resection alone. In another embodiment, peptide agents are administered in conjunction with radiation therapy so as to achieve a better tumorcidal response in the patient than would be presented by radiation treatment alone. Further peptide agents can be administered in conjunction with chemotherapeutic agents. Additionally, peptide agents can be administered in conjunction with radioimmunotherapy so as to treat cancer more effectively than would occur by radioimmunotherapy treatment alone. Still further, peptide agents of the invention can be administered in conjunction with antiviral agents, or agents used to treat Alzheimer's disease.

In some preferred embodiments, therapeutic agents comprising the peptide agents are administered in conjunction with other therapeutic agents that treat viral infections, such as HIV infection, so as to achieve a better viral response. At present four different classes of drugs are in clinical use in the antiviral treatment of HIV-1 infection in humans. These are (i) nucleoside analogue reverse transcriptase inhibitors (NRTIs), such as zidovidine, lamivudine, stavudine, didanosine, abacavir, and zalcitabine; (ii) nucleotide analogue reverse transcriptase inhibitors, such as adetovir and pivaxir; (iii) non-nucleoside reverse transcriptase inhibitors (NNRTIs), such as efavirenz, nevirapine, and delavirdine; and (iv) protease inhibitors, such as indinavir, nelfinavir, ritonavir, saquinavir and amprenavir. By simultaneously using two, three, or four different classes of drugs in conjunction with administration of the peptide agents of the present invention, HIV is less likely to develop resistance, since it is less probable that multiple mutations that overcome the different classes of drugs and the peptide agents will appear in the same virus particle.

It is thus a preferred embodiment of the present invention that peptide agents be given in combination with nucleoside analogue reverse transcriptase inhibitors, nucleotide analogue reverse transcriptase inhibitors, non-nucleoside reverse transcriptase inhibitors, and protease inhibitors at doses and by methods known to those of skill in the art. Medicaments comprising the peptide agents of the present invention and nucleoside analogue reverse transcriptase inhibitors, nucleotide analogue reverse transcriptase inhibitors, non-nucleoside reverse transcriptase inhibitors, and protease inhibitors are also embodiments of the present invention.

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. A method of inhibiting a protein-protein interaction comprising:
identifying a first sequence of a first protein that binds to a second protein, wherein said first sequence is between three and ten consecutive amino acids that bind to said second protein;
providing a peptide agent comprising a peptide in amide form having a second sequence identical to said first sequence; and
contacting said peptide agent with said second protein so as to inhibit said protein-protein interaction.

2. The method of claim 1, wherein said first and second proteins are the same.

3. The method of claim 1, wherein said first and second protein are selected from the group consisting of p24, a bacterial toxin protein, actin, β-amyloid, and tubulin.

4. The method of claim 1, wherein said first protein is a transcriptional activator or transcriptional repressor.

5. The method of claim 1, wherein said first protein is a bacterial toxin protein.

6. The method of claim 1, wherein said first protein is actin.

7. The method of claim 1, wherein said first protein is β-amyloid.

8. The method of claim 1, wherein said first protein is tubulin.

9. A method of inhibiting a protein-protein interaction comprising:
identifying a first sequence of a first protein that binds to a second protein, wherein said first sequence is three consecutive amino acids that bind to said second protein;
providing a tripeptide amide having a second sequence identical to the first sequence; and
contacting said tripeptide amide with said second protein so as to inhibit said protein-protein interaction.

10. The method of claim 9, wherein said first and second proteins are the same.

11. The method of claim 9, wherein said first and second protein are selected from the group consisting of p24, a bacterial toxin protein, actin, β-amyloid, and tubulin.

12. The method of claim 9, further comprising measuring the inhibition of said protein-protein interaction by measuring the binding of said tripeptide amide to said second protein.

13. The method of claim 9, wherein said first protein is a transcriptional activator or transcriptional repressor.

14. The method of claim 9, wherein said first protein is a bacterial toxin protein.

15. The method of claim 9, wherein said first protein is actin.

16. The method of claim 9, wherein said first protein is β-amyloid.

17. The method of claim 7, wherein said first protein is tubulin.

18. A method of making a pharmaceutical comprising:
identifying a first sequence of a first protein that binds to a second protein, wherein said first sequence is between three and ten consecutive amino acids that bind to said second protein; and
providing a peptide agent comprising a peptide in amide form having a second sequence identical to said first sequence.

19. The method of claim 18, wherein said first protein is a transcriptional activator or transcriptional repressor.

20. The method of claim 18, wherein said first protein is a bacterial toxin protein.

21. The method of claim 18, wherein said first protein is actin.

22. The method of claim 18, wherein said first protein is β-amyloid.

23. The method of claim 18, wherein said first protein is tubulin.

24. A method of making a pharmaceutical comprising:
identifying a first sequence of a first protein that binds to a second protein, wherein said first sequence is three consecutive amino acids that bind to said second protein; and
providing a tripeptide amide having a second sequence identical to the first sequence.
